(19) United States
(12) Patent Application Publication
(10) Pub. No.: US 2019/0282600 A1
(43) Pub. Date: Sep. 19, 2019

(54) COMBINATION THERAPIES TARGETING TERT DEPENDENCY FOR CANCER THERAPY

(71) Applicant: The Wistar Institute of Anatomy and Biology, Philadelphia, PA (US)

(72) Inventor: Jesse Villanueva, Hilltown, PA (US)

(21) Appl. No.: 16/354,379

(22) Filed: Mar. 15, 2019

Related U.S. Application Data
(60) Provisional application No. 62/644,267, filed on Mar. 16, 2018.

(51) Int. Cl. A61K 31/7076; A61K 31/675; A61K 45/06; A61P 35/00

(52) U.S. Cl. CPC A61K 31/7076; A61P 35/00; A61K 45/06; A61K 31/675

(57) ABSTRACT

Pharmaceutical compositions and methods for the treatment of cancer are provided. In one embodiment the composition comprises a telomerase inhibitor and an agent that decreases or downregulates anti-oxidant response or induces oxidative stress.

### STATEMENT OF GOVERNMENT INTEREST

This invention was made with government support under Grant Nos. P3OCA010815, R01CA215733, K01CA175269 and P50CA174523 awarded by the National Institutes of Health. The government has certain rights in the invention.

### BACKGROUND

Melanoma is the most aggressive and lethal form of skin cancer. Over the past few years, significant improvement in the treatment of melanoma has been achieved through the use of targeted- and immuno-therapies (Wong and Ribas, 2016). Despite this progress, a large percentage of patients do not benefit from these therapies and/or experience disease progression. In particular, melanomas with neuroblastoma RAS viral oncogene homolog (NRAS) mutations are highly resistant to most therapies and have poor prognosis (Postow and Chapman, 2017; Jakob et al., 2012; Johnson et al., 2014).

Significant improvement in the treatment of melanoma has been achieved through the use of targeted- and immuno-therapies. Despite this progress, a large percentage of patients do not benefit from these therapies and/or experience disease progression. In particular, melanomas with NRAS mutations are highly resistant to most therapies and have poor prognosis.

NRAS is the second most frequently mutated oncogene in melanoma (Hayward et al., 2017; Hodis et al., 2012). In addition to mutations in NRAS, mutations in NF1 (>10%), or activation of receptor tyrosine kinases (RTKs), can also activate RAS signaling in melanoma (Easty et al., 2011; Maertens et al., 2013; Nissan et al., 2014). Further, a frequent mechanism of acquired resistance to BRAF/MEK inhibitors is mediated by secondary mutations in NRAS (Nazarian et al., 2010; Van Allen et al., 2014). Consequently, approximately 40% of melanoma patients have tumors that are driven by aberrant NRAS signaling. Targeting RAS has been remarkably challenging; thus far, there are no drugs in the clinic that directly target mutant NRAS. Alternative approaches, including the use of antagonists of RAS effectors, including RAF and PI3K, have had limited success for the treatment of NRAS-driven metastatic melanoma (Postow and Chapman, 2017; Johnson and Puzanov, 2015). Therefore, there is an urgent need to identify vulnerabilities in this tumor type that can be exploited therapeutically.

TERT, the catalytic subunit of telomerase, is a promising therapeutic target for cancer, as it is highly expressed in most tumor cells and seldom expressed in most normal non-transformed cells (Artandi and DePinho, 2010; Jafri et al., 2016). Mutations in the TERT promoter have been identified in >70% of melanomas, constituting the most frequent genetic alteration in these tumors (Hayward et al., 2017; Huang et al., 2013; Horn et al., 2013). These mutations create de novo Ets/TCF (E-twenty six/ternary complex factor) binding sites, enhancing the expression of TERT in these cells (Hayward et al., 2017; Huang et al., 2013). Clinically, BRAF or NRAS mutant melanoma patients whose tumors have TERT promoter mutations have poor overall survival compared to patients with tumors with a non-mutated (wild type; WT) TERT promoter (Griewank et al., 2014). These data suggest that TERT is a key player in melanoma and a compelling therapeutic target. In addition to its canonical role in maintaining telomere length, TERT has been recognized to regulate extra-telomeric processes. For example, TERT has been shown to regulate apoptosis, DNA damage responses, chromatin state, and cellular proliferation. These combined data suggest that TERT-based strategies might have valuable therapeutic effects.

In addition to its canonical role in maintaining telomere length in malignant cells, TERT has been recognized to regulate extra-telomeric tumor-promoting pathways (Bagheri et al., 2006; Hrdlickova et al., 2012; Li and Tergaonkar, 2014; Listerman et al., 2013). For example, TERT has been shown to regulate apoptosis, the DNA damage response, chromatin state, and cellular proliferation (Choi et al., 2008; Koh et al., 2015; ,Masutomi et al., 2005; Sarin et al., 2005; Shin et al., 2004; Smith et al., 2003). These combined data suggest that TERT-based strategies might be extremely valuable and have multiple therapeutic effects. Unfortunately, developing clinically relevant approaches to inhibit TERT has been daunting. Developing clinically relevant approaches to inhibit TERT has been daunting; most TERT inhibitors evaluated thus far target the enzymatic activity of telomerase and rely on critical shortening of telomeres to kill tumor cells; consequently, there is a prolonged lag period, often greater than 4 weeks, for efficacy (Damm et al., 2001; Joseph et al., 2010). This prolonged lag period could constitute a potential disadvantage, as cancer cells can rapidly adapt to pharmacological challenges and become resistant. Additionally, the long duration of treatment could lead to increased toxicity and/or decreased tolerability. Hence, novel TERT-based therapeutic strategies that can elicit relatively rapid and sustained effects could have significant impact on cancer treatment and are needed.

### SUMMARY

The present invention is based in part on the inventor's discovery of a robust dependency of NRAS mutant melanoma on TERT and that the combination therapy of a telomerase inhibitor and an agent that decreases or downregulates anti-oxidant response or induces oxidative stress effectively treats cancer.

In one aspect, a composition comprising an agent that decreases or downregulates TERT and an agent that decreases or downregulates anti-oxidant response or induces oxidative stress is provided. In one embodiment, a composition comprising an agent that blocks the reverse transcriptase activity of telomerase and an agent that decreases or downregulates anti-oxidant response or induces oxidative stress is provided. In one embodiment, the agent that decreases or downregulates TERT or blocks the reverse transcriptase activity of telomerase is 6-Thio-dG. In one embodiment, the that decreases or downregulates TERT is a telomerase inhibitor. In another embodiment, the agent that reduces an anti-oxidant response is a mitochondria-disrupting agent. In another embodiment, the mitochondria disrupting agent is Gamitrinib. In another embodiment, the agent that inhibits an anti-oxidant response is an Hsp90 inhibitor that acts on mitochondrial Hsp90.

In another aspect, a method of treating cancer is provided. The method includes comprising (i) decreasing or downregulating or degrading or inhibiting TERT and (ii) decreasing or downregulating anti-oxidant response or increasing oxidative stress. In another embodiment, the method includes increasing telomere dysfunction and decreasing or downregulating anti-oxidant response or increasing oxidative stress.

Further aspects will be readily apparent based on the description provided herein.

### BRIEF DESCRIPTION OF THE DRAWINGS

FIGS. 1a to 1i show that depletion of TERT induces rapid death of NRAS^{mut} melanoma cells. FIGS. 1a to 1b shows that NRAS^{mut} melanoma cells were transduced with lentiviruses enconding NRAS shRNA (+) or non-targeting empty vector control (−). As presented in FIG. 1a, NRAS levels were assessed by immunoblotting (bottom panel) and TERT mRNA levels were assessed by qRT-PCR. In FIG. 1b, Relative telomerase activity (R.T.A.) was determined by TRAP assays following NRAS depletion in NRAS^{mut} melanoma cells. Negative control (−): primer; positive control (+): HCT116 cells. FIGS. 1c to 1g show that TERT was depleted using two different hairpins (sh1, sh2). Apoptosis was by determined by flow cytometry using the Annexin V analog PSVue in cells transduced with TERT shRNA 10 days post infection (dpi). TERT mRNA levels are shown below the corresponding bar as fold change relative to vector control (FIG. 1c). As presented in FIG. 1d, telomere length was assessed by Southern blotting of terminal restriction fragments (TRF) in NRAS^{mut} cells 10 days after transduction. (−): empty vector, pGIPZ; (+): TERT shRNA. Mean TRF length expressed as kilobase pairs is indicated at the bottom. FIGS. 1e to 1g show that NRAS^{mut} melanoma cells were transduced with TERT shRNA. Telomere dysfunction-induced foci (TIF) was determined 7 dpi by indirect immunofluorescence combined with fluorescence in situ hybridization (FISH). Bottom panel of FIG. 1e, quantification of TIF assays from two independent experiments. Cells were scored as TIF positive when 4 or more γH2AX foci (middle row of the microscopic images shown in the top panel labelled as γH2AX) co-localized with telomere foci (upper row of the microscopic images shown in the top panel labelled as Telemeres) (FIG. 1e). DNA damage was assessed by γH2AX staining by flow cytometry (FIG. 1f). ROS levels were measured by CellRox-Deep Red staining by FACS (FIG. 1g). FIGS. 1h to 1i show that WM3000 cells were transduced with wild type (WT) or catalytically-impaired VYLF1016 (V1016F), VYFL1028 (N1028W) TERT mutant constructs, followed by shRNA-mediated NRAS silencing. Cell death was assessed by FACS measurement of Annexin V and propidium iodide (PI) staining (FIG. 1h). ROS levels were measured by H2DCFDA fluorescence (FIG. 1i). pLKO.1 and pBlast are empty vectors for NRAS shRNA and TERT constructs, respectively. Data represent average of three independent experiments +/−SEM. *p<0.05; **p<0.01; ***p<0.005 determined by unpaired Student's t-test.

FIGS. 2a to 2h show that 6-thio-dG impairs viability of NRAS^{mut} melanoma cells. FIGS. 2a to 2c show NRAS^{mut} melanoma cells (FIG. 2a), BRAF-V600E/NRAS mutant cells derived from melanoma patients resistant to BRAF/MEK inhibitors (FIG. 2b), or non-transformed melanocytes and fibroblasts (FIG. 2c) were treated with 6-thio-dG for 7 days. Cell viability was measured by Alamar Blue assay and calculated relative to DMSO-treated cells. FIG. 2d shows that NRAS^{mut} melanoma cells and non-transformed human fibroblasts were treated with 1 or 5 μM 6-thio-dG for 7 or 14 days. Cell death was assessed by PSVUe staining. Percent of apoptotic cells is shown. FIG. 2e shows that cells were treated with DMSO or 1 μM 6-thio-dG for 14 days and telomere length was assessed by Southern blotting of TRF. Mean TRF length expressed as kilobase pairs is indicated at the bottom. FIGS. 2f to 2g show that cells were treated with 1 μM 6-thio-dG for 7 days. FIG. 2f shows that ROS production was measured by H2DCFDA fluorescence and normalized to DMSO treated cells. FIG. 2g provides a bar graph showing that DNA damage was assessed by γH2AX staining. Data represent average from three independent experiments ±SEM. *p<0.05; **p<0.01; ***p<0.005 in unpaired Student's t-test. As presented in FIG. 2h, mice bearing established WM3000 or M93-047 NRAS^{mut} tumors were treated with vehicle control or 6-thio-dG (2.5 mg/kg i.p; q.d). Tumor volume was measured and average tumor volume (n=4) ±SEM was plotted vs. time.

FIGS. 3a to 3g provides graphs showing that telomere dysfunction enhances oxidative stress leading to upregulation of an antioxidant program. FIGS. 3a to 3d show that NRAS^{mut} melanoma cells were transduced with TERT shRNA (FIGS. 3a, and 3c) or treated with 1 μM 6-thio-dG for 7 days (FIGS. 3b, and 3d). mRNA levels of the indicated genes were quantified by qRT-PCR (FIGS. 3a, and 3b). Mitochondrial superoxide production was measured by MitoSox Red fluorescence (FIGS. 3c, and 3d) and expressed as fluorescence relative to vector or DMSO controls. FIG. 3e shows that NRAS^{mut} melanoma cells were treated with 1 μM of 6-thio-dG for the indicated times and SOD2 protein levels measured by Western blotting. FIG. 3f provides a bar graph showing that SOD2 was silenced using shRNA. Transduced M93-047 cells were treated with DMSO or 1 μM of 6-thio-dG and cell death was assessed by Annexin V/PI straining. As presented in FIG. 3g, M93-047 cells were transduced with a SOD2 lentiviral construct or empty vector control (pLX304). Transduced cells were treated with increasing doses (1, 2.5 and 5 μM) of 6-thio-dG for 7 days and cell death was assessed. Data represent average from three independent experiments ±SEM. *p<0.05; **p<0.01; ***p<0.005 in unpaired Student's t-test.

FIGS. 4a to 4e provides bar graphs showing that induction of telomere and mitochondrial dysfunction triggers cell death in NRAS mutant melanoma. FIG. 4a shows that NRAS^{mut} melanoma cells transduced with TERT shRNA were treated 7 days post infection with 5 μM Gamitrinib (Gam) for 48 h. Cell death was assessed by Annexin V and PI staining. As presented in FIG. 4b, NRAS^{mut} melanoma cells and non-transformed fibroblasts were treated with 1 μM 6-thio-dG for 5 days. At day 5, culture medium was replaced and cells treated with 1 μM 6-thio-dG plus 5 μM Gam for two more days. Cells were stained with Annexin V and PI and percent cell death was determined by flow cytometry. As shown in FIGS. 4c to 4e, cells were treated as in FIG. 4b and mitochondrial ROS levels were measured using Mitosox Red by FACS. Data represent average of three independent experiments ±SEM. P values were calculated using unpaired Student's t-test; * p<0.05; *** p<0.005.

FIGS. 5a to 5e provide bar graphs showing that combination treatment with 6-Thio-dG and Gamitrinib (Gam) triggers death selectively in NRAS mutant tumor cells. NRAS-WT/BRAF-WT melanoma cells (FIG. 5a), NRAS-WT/BRAF-mut melanoma cells (FIG. 5b), NRAS mutant neuroblastoma cells (FIG. 5c), NRAS-WT/KRAS-mutant colon cancer cells (FIG. 5d), and NRAS-WT/KRAS-mutant lung cancer cells were treated with 1 μM 6-thio-dG for 5 days (FIG. 5e). At day 5, culture medium was replaced and cells treated with 1 μM 6-thio-dG plus 5 μM Gam for two more days. Cells were stained with Annexin V and PI and percent cell death was determined by FACS. Data represent average of three independent experiments +/−SEM; p values were calculated using un-paired Student's t-test; *p<0.05; **p<0.001.

FIGS. 6a to 6e provide results showing that combining 6-thio-dG and Gamitrinib inhibits the growth of 3D melanoma spheroids and NRAS-mutant xenograft tumors. Collagen-embedded melanoma spheroids were treated with DMSO, 6-thio-dG (5 μM) and Gam (5 μM) as in FIGS. 4a to 4e. On day 7, spheroids were stained with Calcein AM (live cells; green) and EtBr (dead cells; red) and imaged using an inverted microscope (4X; scale bar=250 μm). Representative merged images from three independent experiments are shown in FIG. 6a. FIGS. 6b to 6d show that NRAS mutant M93-047 tumor bearing mice were treated with vehicle control, 6-thio-dG (2.5 mg/kg; ip qd), Gam (12.5 mg/kg; ip qod), or the combination of the two drugs for the indicated days (n=7 mice/group). As shown in FIG. 6b, average tumor volume over time ±SEM is represented. Average tumor weight ±SEM after 14 days of treatment is presented in FIG. 6c. P values were calculated using unpaired Student's t-test. *p<0.05, ** p<0.01, ***p<0.005, when comparing vehicle control vs. combination treatment groups. As presented in FIG. 6d, treatment was discontinued after 14 days, and mice were followed until tumor volume reached a preset volume (1500 mm³). Kaplan Meier survival curves of mice treated for 14 days. p-values were calculated by Mantel-Cox log-rank test; *p<0.05, **p<0.01, ***p<0.005. Animal weight (grams) before treatment (Start of TX) and at the end of the study (End of TX) was assessed, recorded for every mouse enrolled in the study and plotted in FIG. 6e. Mean and SEM are depicted (n=7 mice/treatment group). Statistical significance was assessed by unpaired Student's t-test.

FIG. 7 provides an illustration showing that TERT offsets oxidative stress in NRAS mutant melanoma. TERT is required for telomere integrity. Genetic silencing of TERT or pharmacological induction of telomere uncapping due to incorporation of the nucleoside analogue 6-thio-dG triggers rapid telomere-induced foci (TIF) coupled to global DNA damage and increased production of ROS. Enhanced ROS levels prompts the activation of a ROS scavenging adaptive response, mediated mainly by SOD2, which re-establishes steady levels of ROS and offsets oxidative stress. Treatment of NRAS mutant melanoma cells with Gamitrinib, a mitochondrial disrupting agent, attenuates SOD2 levels impairing its ability to effectively restore redox balance, resulting in toxic levels of ROS and tumor cell death.

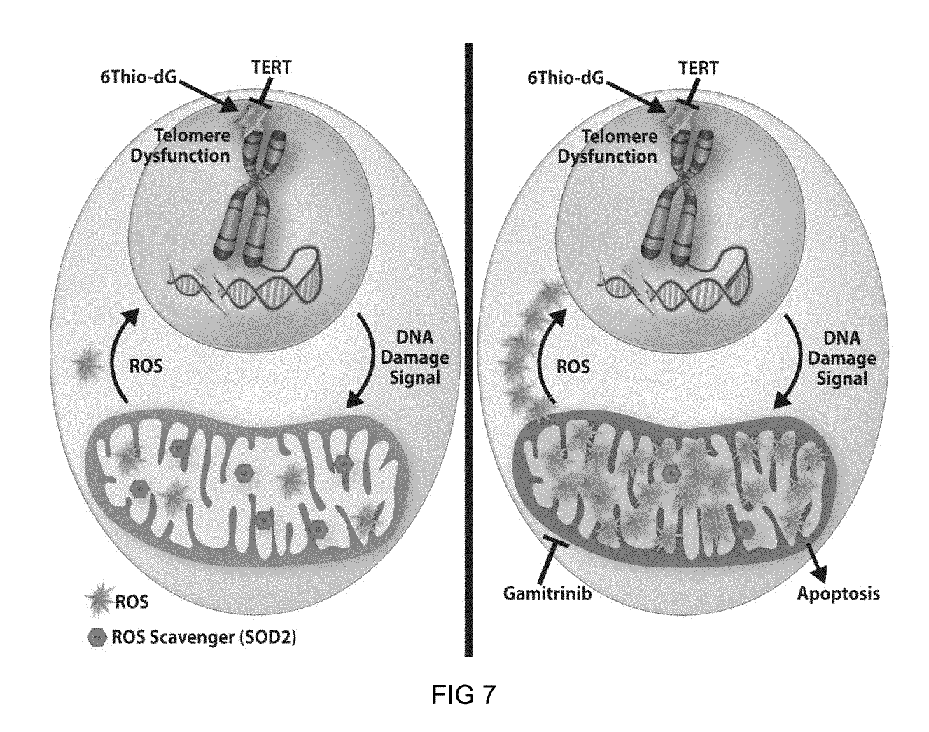

FIG. 8 shows that NRAS silencing is coupled to downregulation of TERT. NRAS-mutant WM3000 cells transduced with lentiviruses encoding NRAS shRNA were analyzed using the human cellular senescence PCR array profiler. mRNA levels of the indicated genes were assessed by qRT-PCR following NRAS knockdown.

FIGS. 9a to 9d show that MEK inhibition downregulates TERT levels. NRAS mutant melanoma cells WM3000 (FIGS. 9a, and 9b) or WM3629 (FIGS. 9c, and 9d) were treated with the MEK inhibitor trametinib (100 nM) for 24, 48, 72 h. Inhibition of phospho-ERK was assessed by immunoblotting. Dotted lines indicated where membranes were cut to remove non-relevant lanes (FIGS. 9a, and 9c). Relative expression of TERT mRNA levels was determined by qRT-PCR (FIGS. 9b, and 9d). Data represent average of triplicates +/−SD.

FIGS. 10a to 10b show that inhibition of cdk4/6 transiently attenuates TERT expression. NRAS mutant melanoma cells WM3000 were treated with the cdk4/6 inhibitor palbociclib at the indicated doses for 72 h. As presented in FIG. 10a, downregulation of phospho-Rb and cyclin A was evaluated by immunoblotting as a surrogate of cell cycle arrest. In FIG. 10b, relative expression of TERT mRNA levels was determined by qRT-PCR. Data represent average of triplicate samples.

FIGS. 11a to 11c provides that TERT depletion triggers apoptosis. As shown in FIGS. 11a to 11c, NRAS^{mut} melanoma cell lines were transduced with lentiviruses encoding TERT shRNA using two different hairpins (sh1, sh2) or non-targeting empty vector pGIPZ and analyzed 11 days post infection (dpi). Data shown represent average of three independent experiments +/−SEM. TERT mRNA levels were assessed by qRT-PCR (FIG. 11a). Apoptosis was determined using cleaved caspase 3 by FACS (FIG. 11b). Apoptosis was also assessed by using the Annexin V analog PSVue and senescence was assessed using the ImaGene Red β-galactosidase substrate C12RG by FACS (FIG. 11c).

FIGS. 12a to 12b show that TERT silencing triggers rapid telomere dysfunction-induced foci. As shown in FIG. 12a, telomere induced foci (TIF) was determined by indirect immunofluorescence combined with fluorescence in situ hybridization (FISH) in NRAS^{mut} cells transduced with TERT shRNA 7 dpi. Cells were considered TIF positive when four or more co-localizing foci of telomeres (red, top row of FIG. 12a labeled as Telomeres) and 53BP1 (green, middle row of FIG. 12a labeled as 53BP1) were found. Quantification of TIF positive cells from two independent experiments is shown in FIG. 12b. Data are average of two independent experiments +/−SEM.

FIGS. 13a to 13b show that treatment with the antioxidant N-acetyl-L-cysteine (NAC) attenuates TIFs. NRAS mutant melanoma cells WM3000 were transduced with TERT shRNA and treated with NAC (mM) for 7 days. As shown in FIG. 13a, TIFs were assessed by indirect immunofluorescence combined with fluorescence in situ hybridization (FISH). Representative images are shown. (scale bar=10 μm). Quantification of TIF positive cells is plotted in FIG. 13b. Cells were scored as TIF positive when three or more g-H2AX foci (green) were co-localizing with telomere foci (red). Data represent average of thirteen fields imaged +/−SD.

FIGS. 14a to 14b show that catalytically impaired TERT mutants lead to telomere attrition in NRAS mutant melanoma cells. As presented in FIG. 14a, telomere length was assessed by Southern blotting of terminal restriction fragments (TRF) in WM3000 cells transduced with vector control, wild type (WT), or catalytic-impaired hTERT mutant constructs VYLF1016 (V16F) or VYFL1028 (N28W). In FIG. 14b, relative cell viability was determined by MTT assays at days 1, 3, 6, 8 and 10 post-transduction. Cell viability was calculated relative to vector control-transduced cells.

FIGS. 15a to 15d show that NRAS mutant melanoma cells are sensitive to the telomere uncapping agent 6-thio-dG. NRAS^{mut} melanoma cells were treated with 6-thio-dG for 6, 12 or 18 days. As presented in FIG. 15a, relative cell number was assessed by Crystal Violet assay and calculated relative to DMSO-treated cells. Data represent average of three independent experiments +/−SD. FIG. 15b provides representative pictures of cells treated with 6-thio-dG for the indicated days and stained with Crystal Violet. NRAS^{mut} cells were treated with 6-thio-dG for 7 days and telomere induced foci (TIF) was determined by indirect immunofluorescence combined with fluorescence in situ hybridization (FISH) (FIG. 15c). Cells were considered TIF positive when four or more co-localizing foci of telomeres and g-H2AX were found (FIG. 15c). Quantification of TIF assay is plotted in FIG. 15d. Cells were scored as TIF positive when four or more γ-H2AX foci (green, center row of FIG. 15c labelled as γH2AX) were co-localizing with telomere foci (red, top row of FIG. 15c labelled as Telomeres). Data represent average of two independent experiments +/−SEM. P values were calculated by unpaired Student's t-test.

FIGS. 16a to 16b show that SOD2 modulates the response to 6-thio-dG. As presented in FIG. 16a, SOD2 was silenced using shRNA. Transduced WM3000 cells were treated with DMSO or 1 μM of 6-thio-dG and cell death was assessed by Annexin V/PI straining. In FIG. 16b, cells were transduced with a SOD2 lentiviral construct or empty vector control (pLX304). Transduced cells were treated with increasing doses (1, 2.5 and 5 μM) of 6-thio-dG for 10 days and cell death was assessed. Data represent average of three independent experiments +/−SEM. *p<0.05 in unpaired Student's t-test.

FIGS. 17a to 17b show that impairing mitochondrial function potentiates the effects of 6-thio-dG. In FIG. 17a, NRAS^{mut} melanoma cells were treated with 6-thio-dG (1 μM) for 7 days total. At day 5, culture medium was replaced and cells were treated with 1 μM 6-thiodG plus 1 mM Phenformin for two more days. Percent cell death was determined by flow cytometry following staining of cells with Annexin V and Propidium iodide. Data represent average of three independent experiments +/−SEM. *p<0.05 in unpaired Student t test. In FIG. 17b, collagen-embedded spheroids were treated with DMSO, 6-thio-dG (5 μM) and Ganetespib (5 μM) as in FIG. 17a. On day 7, spheroids were stained with Calcein-AM (live cells; green) and EtBr (dead cells; red) and imaged using an inverted microscope (4×). Representative merged images from three independent experiments are shown.

### DETAILED DESCRIPTION OF THE INVENTION

Mutations in NRAS mutant are present in nearly 30% of melanoma patients. Targeting NRAS directly has not been possible thus far, consequently patients whose tumors harbor NRAS mutations have limited therapeutic options and poor prognosis. Therefore, there is an unmet need to identify novel targets or tumor vulnerabilities that can be therapeutically exploited. In this study, we investigated the consequences of NRAS silencing in NRAS mutant melanoma. We show that NRAS silencing, led to decreased TERT expression and activity. We further show that genetic inhibition or pharmacological blockade of TERT leads to rapid apoptosis of NRAS-mutant melanoma cells. We found that treatment of NRAS-mutant melanoma cells with the telomere uncapping agent 6-Thio-dG led to telomeric and non-telomeric DNA damage, apoptosis and slower tumor growth rate. Telomere dysfunction was coupled to increased reactive oxygen species (ROS) levels and up-regulation of a mitochondrial anti-oxidant response. Furthermore, combining 6-Thio-dG with the mitochondrial disrupting agent Gamitrinib, triggered redox imbalance, enhanced suppression of NRAS mutant melanoma, and significantly prolonged mouse survival.

### I. DEFINITIONS

Technical and scientific terms used herein have the same meaning as commonly understood by one of ordinary skill in the art to which this invention belongs and by reference to published texts, which provide one skilled in the art with a general guide to many of the terms used in the present application. The following definitions are provided for clarity only and are not intended to limit the claimed invention.

The terms “a” or “an” refers to one or more, for example, “a Gamitrinib” is understood to represent one or more Gamitrinib compounds. As such, the terms “a” (or “an”), “one or more,” and “at least one” are used interchangeably herein. As used herein, the term “about” means a variability of 10% from the reference given, unless otherwise specified. While various embodiments in the specification are presented using “comprising” language, under other circumstances, a related embodiment is also intended to be interpreted and described using “consisting of” or “consisting essentially of” language.

“Patient” or “subject” as used herein means a mammalian animal, including a human, a veterinary or farm animal, a domestic animal or pet (including cats and dogs), and animals normally used for clinical research (including mice, rats, non-human primates, etc). In one embodiment, the subject of these methods and compositions is a human. In a further embodiment, the subject of these methods and compositions is a human having cancer.

The term “cancer” or “proliferative disease” as used herein means any disease, condition, trait, genotype or phenotype characterized by unregulated cell growth or replication as is known in the art. A “cancer cell” is cell that divides and reproduces abnormally with uncontrolled growth. This cell can break away from the site of its origin (e.g., a tumor) and travel to other parts of the body and set up another site (e.g., another tumor), in a process referred to as metastasis. A “tumor” is an abnormal mass of tissue that results from excessive cell division that is uncontrolled and progressive, and is also referred to as a neoplasm. Tumors can be either benign (not cancerous) or malignant. The methods described herein are useful for the treatment of cancer and tumor cells, i.e., both malignant and benign tumors, so long as the cells to be treated have mitochondrial localization of the chaperones as described herein. In various embodiments of the methods and compositions described herein, the cancer can include, without limitation, breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, acute and chronic lymphocytic and myelocytic leukemia, myeloma, Hodgkin's and non-Hodgkin's lymphoma, and multidrug resistant cancer. In one embodiment, the cancer is a melanoma. In another embodiment, the cancer is a NRAS mutant cancer. Such NRAS mutant cancers include, without limitation, somatic rectal cancer, follicular thyroid cancer, autoimmune lymphoproliferative syndrome, Noonan syndrome, juvenile myelomonocytic leukemia, giant congenital melanocytic nevus, core binding factor acute myeloid leukemia, cytogenetically normal acute myeloid leukemia, lung cancer, and melanoma. Other NRAS mutant cancers include melanoma, thyroid, endometrial, non-small cell lung, ovarian and colorectal cancers. See, e.g., Madhusudanannair et al, Journal of Clinical Oncology 30, no. 15_suppl (May 20 2012) 3106-3106, which is incorporated herein by reference. In another embodiment, the cancer is NRAS mutant melanoma. In another embodiment, the cancer is NRAS mutant brain cancer. In another embodiment, the cancer is NRAS mutant neuroblastoma.

As used herein, the term “any intervening amount”, when referring to a range includes any number included within the range of values, including the endpoints.

The term “regulation” or variations thereof as used herein refers to the ability of a compound of a compound or composition described herein to increase or inhibit one or more components of a biological pathway. In certain embodiments, the terms “increase”, “upregulate” or any variation thereof refers to about 105%, about 110%, about 120%, about 130%, about 140%, about 150%, about 160%, about 170%, about 180%, about 190%, about 2-fold, about 5-fold, or more of the reference given, unless otherwise specified. In certain embodiments, the terms “decrease” “downregulate” “inhibit” or any variation thereof refers to about 5%, about 10%, about 20%, about 30%, about 40%, about 50%, about 60%, about 70%, about 80%, about 90%, about 95% of the reference given, unless otherwise specified.

As used herein, “disease”, “disorder” and “condition” are used interchangeably, to indicate an abnormal state in a subject. In certain embodiment, disease refers to a cancer. The term “treating” or “treatment” is meant to encompass administering to a subject a compound described herein for the purposes of amelioration of one or more symptoms of a disease or disorder.

The recent discovery of TERT promoter mutations in the majority of melanomas has provided compelling evidence that this gene plays a critical role in this disease, and could therefore constitute a promising therapeutic target. As described herein, NRAS mutant melanoma was used as a model to investigate the therapeutic value of exploiting melanoma's addiction to TERT. In addition, mechanisms limiting the effects of TERT-based approaches were investigated to develop rational combinations that can improve their efficacy. It was found that telomere induced dysfunction is associated with oxidative damage followed by relatively rapid melanoma cell death in vitro and decreased tumor growth in vivo. However, tumor cells might counteract these effects by activating an anti-oxidant program aimed at restoring redox balance and resisting the detrimental effects of excessive ROS levels. Based on these results, it is believed that TERT blockade or 6-Thio-dG induces telomere dysfunction and prompts the activation of a detoxifying program aimed at buffering excessive ROS levels. Impairing mitochondrial function and blunting the ROS scavenging machinery renders tumor cells susceptible to excessive ROS levels, leading to tumor cell death. By combining agents that induce telomere dysfunction, such as 6-Thio-dG, with an oxidative stress inducing agent, such as the mitochondriotoxic inhibitor Gamitrinib, NRAS mutant tumors can be restrained, increasing the survival of NRAS mutant tumor-bearing mice.

Approximately 30-40% of melanomas are driven by aberrant NRAS signaling and are highly resistant to most therapies. Therefore, there is an urgent unmet need to identify new targets and develop effective therapies for NRAS mutant tumors. The reverse transcriptase TERT is a compelling target for cancer therapy, as it is highly expressed in most tumors, while seldom expressed in adult tissues. Consistent with these studies, TERT promoter mutations render TERT expression dependent on MAPK signaling (Li et al., 2016; Vallarelli et al., 2016). It was found that NRAS silencing decreased TERT expression and activity in NRAS mutant melanoma cells with either wild-type, or to a lesser extent, mutant TERT promoter. These data substantiate the contribution of RAS/MAPK signaling for TERT expression and function, underscoring the potential value of TERT-based approaches for melanoma therapy.

### II. COMPOSITIONS

In one aspect, pharmaceutical compositions are provided. In one aspect, the pharmaceutical composition comprises an agent that decreases or downregulates TERT or degrading or inhibiting or blocks the reverse transcriptase activity of telomerase and a compound that induces oxidative stress or reduces antioxidant response. In one embodiment, the composition is a combination of the listed therapeutic agents, which are not necessarily combined into a single dosage format. As used herein, when referring to a “composition”, it is intended that a similar embodiment is encompassed which refers to a pharmaceutical “combination” as noted above.

In one embodiment, the agent that reduces anti-oxidant response is a mitochondria targeting agent. As used herein, mitochondria targeting agent refers to an agent which inhibits the function of mitochondria. Specifically, in one embodiment, such agent is selective for cancer cells. Such agents include those which permeabilize mitochondria and those which induce apoptosis. Such agents include small molecule compounds and peptides. In one embodiment, the mitochondria disrupting agent is a peptide, such as a penetratin-directed peptide. Yang et al, J. Biological Chem., 2010, 285(33):25666-76 describe such peptides, which include an Antp leader peptide (i.e., a mitochondria-penetrating moiety), fused with a mitochondria-disrupting peptide (e.g., KGA, BA27 or BA28).

| (SEQ ID NO: 1) | |
|---|---|
| Antp: | KKWKMRRNQFWVKVQRG. |
| (SEQ ID NO: 2) | |
| KGA: | KLAKLAKKLAKLAKGGKKWKMRRNQFWVKVQRG |
| (SEQ ID NO: 3) | |
| BA27: | GRFKRFRKKFKKLFKKLSKKWKMRRNQFWVKVQRG |
| (SEQ ID NO: 4) | |
| BA28: | GGLRSLGRKILRAWKKYGGKKWKMRRNQFWVKVQRG. |

In one embodiment, the mitochondria disrupting agent is an Hsp70 inhibitor. In one embodiment, the HSP70 inhibitor is VER-155008. In another embodiment, the Hsp70 inhibitor is 2-phenylethynesulfonamide (PES). In another embodiment, the Hsp70 inhibitor is JG-98 or a derivative thereof. Other suitable Hsp70 inhibitors include, without limitation, 115-7c, apoptozole, JG-13, JG-48, MAL3-101, methylene blue, MKT-077, pifithrin-mu, spergualin, YM-01 and YM-08.

In one embodiment, the mitochondria disrupting agent is Gamitrinib. Gamitrinib is a molecule that inhibits selectively the pool of Hsp90 localized to mitochondria of tumor cells. As used herein, the term “Gamitrinib” refers to any one of a class of geldanamycin (GA)-derived mitochondrial matrix inhibitors. Gamintrinibs contain a benzoquinone ansamycin backbone derived from the Hsp90 inhibitor 17-(allylamino)-17-demethoxygeldanamycin (17-AAG), a linker region on the C17 position, and a mitochondrial targeting moiety, either provided by 1 to 4 tandem repeats of cyclic guanidinium (for example, a tetraguanidinium (G4), triguanidinium (G3), diguanidinium (G2), monoguanidinium (G1),) or triphenylphosphonium moiety (Gamitrinib-TPP-OH). For example, Gamitrinib-G4 refers to a Gamitrinib in which a tetraguanidinium moiety is present. For example, Gamitrinib-TPP refers to a Gamitrinib in which a triphenylphosphonium moiety is present. Also, throughout this application, the use of the plural form “Gamitrinibs” indicates one or more of the following: Gamitrinib-G4, Gamitrinib-G3, Gamitrinib-G2, Gamitrinib-G1, and Gamitrinib-TPP or Gamitrinib-TPP-OH. Gamitrinib is a small molecule inhibitor of Hsp90 and TRAP-1 ATPase activity, engineered to selectively accumulate in mitochondria. In one embodiment, the Gamintrinib is Gamitrinib-PP-OH. In another embodiment, the Gamitrinib is Gamitrinib-G4. The approximate molecular weights of the Gamitrinibs discussed herein are the following: Gamitrinib-Gl: 1221.61 g/mol; Gamitrinib-G2: 709.85 g/mol; Gamitrinib-G3: 539.27 g/mol; Gamitrinib-G4: 604.97g/mol; and Gamitrinib-TPP: 890.46 g/mol. See, e.g., United States Patent Publication No. 2009/0099080 and Kang et al, 2009, J. Clin. Invest, 119(3):454-64 (including supplemental material), which are hereby incorporated by reference in their entirety.

The terms “mitochondria-penetrating moiety” and “mitochondria-targeting moiety” are used herein interchangeably. In one embodiment, by “mitochondria-penetrating moiety” or “mitochondria-targeting moiety” it is meant a molecule that targets to and, together with its cargo, accumulates in mitochondria due to its: i) high affinity binding to one or more of intra-mitochondrial sites, ii) hydrophobicity and positive charge, iii) ability to enter mitochondria via carrier proteins unique to the organelle, and iv) specific metabolism by mitochondrial enzymes. In another embodiment, by “mitochondria-penetrating moiety” or “mitochondria-targeting moiety” it is meant a molecule which utilizes “electrophoresis” of the vehicle and cargo into mitochondria at the expense of negative inside membrane potential. See, e.g., Belikova et al, FEBS Lett. 2009 June 18; 583(12): 1945-1950 and U.S. Patent Publication No. 2009/0099080.

In another embodiment, the mitochondria-targeting agent is phenformin. In another embodiment, the mitochondria-targeting agent is Pet-16. In another embodiment, the mitochondria-targeting agent is Lonidamine. In another embodiment, the mitochondria-targeting agent is Betulinic Acid. In another embodiment, the mitochondria-targeting agent is GSAO ((4-[N-[S-glutathionylacetyl]amino] phenylarsenoxide). In another embodiment, the mitochondria-targeting agent is a Retinoid-related compound (CD437 and the alltrans-retinoic acid, 9-cis-retinoic acid and 13-cis-retinoic acids). In another embodiment, the mitochondria-targeting agent is Motexafin gadolinium (gadolinium texaphyrin). In another embodiment, the mitochondria-targeting agent is Menadione (2-methyl-1,4-naphthoquinone) or a thiol crosslinking agent such as diamide (diazenediacarboxylic acid bis 5N,N-dimethylamide), bismaleimido-hexane (BMH) and dithiodipyridine (DTDP). In another embodiment, the mitochondria-targeting agent is Beta-lapachone. In another embodiment, the mitochondria-targeting agent is Butathione sulphoximine. In another embodiment, the mitochondria-targeting agent is Elesclomol sodium. In another embodiment, the mitochondria-targeting agent is imexon. In another embodiment, the mitochondria-targeting agent is ABT-737. In another embodiment, the mitochondria-targeting agent is metformin. In another embodiment, the mitochondria-targeting agent is ABT-263. In another embodiment, the mitochondria-targeting agent is Gossypol. In another embodiment, the mitochondria-targeting agent is A-385358. In another embodiment, the mitochondria-targeting agent is Obatoclax. In another embodiment, the mitochondria-targeting agent is 3-bromopyruvate. In another embodiment, the mitochondria-targeting agent is 2-deoxy-d-glucose.

In another embodiment, the agent that reduces anti-oxidant response is a Hsp90 inhibitor that targets mitochondrial Hsp90 or TRAP1. One such inhibitor includes Gamitrinib, as discussed above. Hendriks & Dingemans, Expert Opinion on Investigational Drugs, 26(5):541-50 (Mar 2017), describes Hsp antagonists suitable herein. Such document is incorporated herein by reference. In one embodiment, the Hsp90 inhibitor is SMTIN-P01. See, Lee et al, J. Am. Chem. Soc., 2015, 137 (13), pp 4358-4367, which is incorporated herein by reference. Other suitable Hsp90 inhibitors are known in the art and are useful herein. See, e.g., Wang et al, J. Med. Chem., 59, 12, 5563-5586 (Feb. 2016), which is incorporated herein by reference.

In some embodiments of the compositions and methods described herein, the agent which decreases or downregulates or degrading or inhibiting TERT or blocks the reverse transcriptase activity of telomerase includes telomerase inhibitors. Telomerase is a ribonucleoprotein that maintains the lengths of chromosomal ends by synthesizing telomeric sequences. As used herein, the term “telomerase inhibitor” specifically includes 6-thio-dG, a telomerase uncapping agent. See, Mender et al, Oncoscience 2015, 2, 8, which is incorporated herein by reference. Telomerase is central to cellular immortality and is a key component of most cancer cells although this enzyme is rarely expressed to significant levels in normal cells. Therefore, the inhibition of telomerase has garnered considerable attention as a possible anticancer approach. Many of the methods applied to telomerase inhibition focus on either of the two major components of the ribonucleoprotein holoenzyme, that is, the telomerase reverse transcriptase (TERT) catalytic subunit or the telomerase RNA (TR) component. Other protocols have been developed to target the proteins, such as tankyrase, that are associated with telomerase at the ends of chromosomes.

In one embodiment, the telomerase inhibitor is any agent that leads to telomere dysfunction. In one embodiment, the telomerase inhibitor is 6-thio-2′-deoxyguanosine (6-thio-dG). See Nature Reviews Cancer volume 16, pages 508-524 (2016) doi:10.1038/nrc.2016.55 and Mender et al, Cancer Discov. 2015 Jan;5(1):82-95. Epub 2014 Dec. 16, which are incorporated herein by reference. Other nucleoside analogs are useful herein. In one embodiment, the telomerase inhibitor is zidovudine (also known as azidothymidine). In another embodiment, the telomerase inhibitor is stavudine. In another embodiment, the telomerase inhibitor is tenofovir. In another embodiment, the telomerase inhibitor is didanosine. In another embodiment, the telomerase inhibitor is abacavir. In another embodiment, the telomerase inhibitor is rhodocyanine (also known as FJ5002). In another embodiment, the telomerase inhibitor is EGCG or pro-EGCG. In another embodiment, the telomerase inhibitor is MST-312 (an EGCG derivative). In another embodiment, the telomerase inhibitor is curcumin. In another embodiment, the telomerase inhibitor is genistein. In another embodiment, the telomerase inhibitor is TMPI. In another embodiment, the telomerase inhibitor is telomestatin. In another embodiment, the telomerase inhibitor is RHPS4. In another embodiment, the telomerase inhibitor is BRACO-19. In another embodiment, the telomerase inhibitor is TMPyP4. In another embodiment, the telomerase inhibitor is tertomotide. In another embodiment, the telomerase inhibitor is imetelstat sodium. In another embodiment, the telomerase inhibitor is ASTVAC-1. In another embodiment, the telomerase inhibitor is GX-301. In another embodiment, the telomerase inhibitor is UCPVax. In another embodiment, the telomerase inhibitor is UV-1. In another embodiment, the telomerase inhibitor is Vx-001. In another embodiment, the telomerase inhibitor is Vx-006. In another embodiment, the telomerase inhibitor is INO-1400. In another embodiment, the telomerase inhibitor is INVAC-1. In another embodiment, the telomerase inhibitor is ASTVAC-2. In another embodiment, the telomerase inhibitor is Telin. In another embodiment, the telomerase inhibitor is Vbx-011. In another embodiment, the telomerase inhibitor is Vbx-021. In another embodiment, the telomerase inhibitor is Vbx-026. In another embodiment, the telomerase inhibitor is INO-5401. In another embodiment, the telomerase inhibitor is KML-001. In another embodiment, the telomerase inhibitor is TK-005. In another embodiment, the telomerase inhibitor is Ribovax. In another embodiment, the telomerase inhibitor is Vbx-016. In another embodiment, the telomerase inhibitor is ZI-HX. In another embodiment, the telomerase inhibitor is ZI-H04. In another embodiment, the telomerase inhibitor is ZIH-03.

A pharmaceutical composition comprising a telomerase inhibitor and inhibitor of antioxidant response described herein is generally formulated to be compatible with its intended route of administration. In one embodiment, the composition is formulated as two separate components. In another embodiment, the composition contains all components in a single formulation. In one embodiment, the composition includes a pharmaceutically acceptable carrier or diluent. Examples of routes of administration include parenteral, e.g., intravenous, intradermal, subcutaneous, inhalation (oral, tranasal, and intratracheal), ocular, transdermal (topical), subligual, intracrainial, epidural, vaginal, intraperitoneal, intratumoral, intranodal, transmucosal, and rectal administration. Routes of administration may be combined, if desired. The route of administration for each component may be different or the same. In some embodiments, the administration is repeated periodically.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as EDTA; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions, and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be achieved by including an agent which delays absorption, e.g., aluminum monostearate or gelatin, in the composition.

Sterile injectable solutions can be prepared by incorporating an active compound (e.g., telomerase inhibitor and/or inhibitor of antioxidant response) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Although the composition may be administered alone, it may also be administered in the presence of one or more pharmaceutical carriers that are physiologically compatible. The carriers may be in dry or liquid form and must be pharmaceutically acceptable. Liquid pharmaceutical compositions are typically sterile solutions or suspensions. When liquid carriers are utilized for parenteral administration, they are desirably sterile liquids. Liquid carriers are typically utilized in preparing solutions, suspensions, emulsions, syrups and elixirs. In one embodiment, the composition may be combined with a liquid carrier. In another embodiment, the composition may be suspended in a liquid carrier. One of skill in the art of formulations would be able to select a suitable liquid carrier, depending on the route of administration. The composition may alternatively be formulated in a solid carrier. In one embodiment, the composition may be compacted into a unit dose form, i.e., tablet or caplet. In another embodiment, the composition may be added to unit dose form, i.e., a capsule. In a further embodiment, the composition may be formulated for administration as a powder. The solid carrier may perform a variety of functions, i.e., may perform the functions of two or more of the excipients described below. For example, the solid carrier may also act as a flavoring agent, lubricant, solubilizer, suspending agent, filler, glidant, compression aid, binder, disintegrant, or encapsulating material.

Oral compositions generally include an inert diluent or an edible carrier. For the purpose of oral therapeutic administration, the active compound can be incorporated with excipients and used in the form of tablets, troches, gel tab, dispersible powder, granule, suspension, liquid, thin film, chewable tablet, rapid dissolve tablet, medical lollipop, or fast melt or capsules, e.g., gelatin capsules. Oral compositions can also be prepared using a fluid carrier for use as a mouthwash. One of skill in the art would readily be able to formulate the compositions discussed herein in any one of these forms.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL™, or corn starch; a lubricant such as magnesium stearate; a glidant such as colloidal silicon dioxide; a sweetening agent such as sucrose or saccharin; syrup; coloring agent; coating; emulsifier; emollient; encapsulating material; granulating agent; metal chelator; osmo-regulator, pH adjustor; preservative; solubilizer; sorbent; stabilizer; surfactant; suspending agent; thickener; viscosity regulator; or a flavoring agent such as peppermint, methyl salicylate, or orange flavoring. See, for example, the excipients described in the “Handbook of Pharmaceutical Excipients”, 5^{th} Edition, Eds.: Rowe, Sheskey, and Owen, APhA Publications (Washington, D.C.), Dec. 14, 2005, which is incorporated herein by reference.

For administration by inhalation, a compound is delivered in the form of an aerosol spray from pressured container or dispenser that contains a suitable propellant, e.g., a gas or liquified propellant, e.g., dichlorodifluoromethane, such as carbon dioxide, nitrogen, propane and the like or a nebulizer. Also provided is the delivery of a metered dose in one or more actuations.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In a further embodiment, the composition may be administered by a sustained delivery device. “Sustained delivery” as used herein refers to delivery of the composition which is delayed or otherwise controlled. Those of skill in the art are aware of suitable sustained delivery devices. For use in such sustained delivery devices, the composition is formulated as described herein. In one embodiment, the compounds may be formulated with injectable microspheres, bio-erodible particles, polymeric compounds (polylactic or polyglycolic acid), beads, liposomes, or implantable drug delivery devices.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

As discussed above, compositions useful herein contain a telomerase inhibitor and an agent that decreases or downregulates anti-oxidant response or induces oxidative stress in a pharmaceutically acceptable carrier optionally with other pharmaceutically inert or inactive ingredients. In another embodiment, a telomerase inhibitor and an agent that decreases or downregulates anti-oxidant response or induces oxidative stress are present in multiple compositions. In another embodiment a telomerase inhibitor and an agent that decreases or downregulates anti-oxidant response or induces oxidative stress are present in a single composition. In a further embodiment, a telomerase inhibitor and an agent that decreases or downregulates anti-oxidant response or induces oxidative stress inhibitor are combined with one or more excipients and/or other therapeutic agents as described below.

The composition(s) may be administered on regular schedule, i.e., daily, weekly, monthly, or yearly basis or on an irregular schedule with varying administration days, weeks, months, etc. Alternatively, administration of the composition may vary. In one embodiment, the first dose of the composition is higher than the subsequent doses. In another embodiment, the first dose containing the composition is lower than subsequent doses. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The composition may be formulated neat or with one or more pharmaceutical carriers for administration. The amount of the pharmaceutical carrier(s) is determined by the solubility and chemical nature of the components of the composition, chosen route of administration and standard pharmacological practice. The pharmaceutical carrier(s) may be solid or liquid and may include both solid and liquid carriers. A variety of suitable liquid carriers is known and may be selected by one of skill in the art. Such carriers may include, e.g., DMSO, saline, buffered saline, hydroxypropylcyclodextrin, and mixtures thereof. Similarly, a variety of solid carriers and excipients are known to those of skill in the art.

As used herein, the term “effective amount” or “pharmaceutically effective amount” as it refers to individual composition components, refers to the amount of a telomerase inhibitor and /or an agent that decreases or downregulates anti-oxidant response or induces oxidative stress described herein that elicits the biological or medicinal response in a tissue, system, animal, individual or human that is being sought by a researcher, veterinarian, medical doctor or other clinician, which includes one or more of the following, preventing a disease; e.g., inhibiting a disease, condition or disorder in an individual that is experiencing or displaying the pathology or symptomatology of the disease, condition or disorder (i.e., arresting or slowing further development of the pathology and/or symptomatology); ameliorating a disease, condition or disorder in an individual that is experiencing or displaying the pathology or symptomatology of the disease, condition or disorder (i.e., reversing the pathology and/or symptomatology); and inhibiting a physiological process. For example, an effective amount of a combination of a telomerase inhibitor and an agent that decreases or downregulates anti-oxidant response or induces oxidative stress, when administered to a subject to treat cancer, is sufficient to inhibit, slow, reduce, or eliminate tumor growth in a subject having cancer.

The effective dosage or amount of the compounds may vary depending on the particular compound employed, the mode of administration, the type and severity of the condition being treated, and subject being treated as determined by the subject's physician. The effective dosage of each active component (e.g., a telomerase inhibitor and an agent that decreases or downregulates anti-oxidant response or induces oxidative stress) is generally individually determined, although the dosages of each compound can be the same. In one embodiment, the dosage is about 1 μg to about 1000 mg. In one embodiment, the effective amount is about 0.1 to about 50 mg/kg of body weight including any intervening amount. In another embodiment, the effective amount is about 0.5 to about 40 mg/kg. In a further embodiment, the effective amount is about 0.7 to about 30 mg/kg. In still another embodiment, the effective amount is about 1 to about 20 mg/kg. In yet a further embodiment, the effective amount is about 0.001 mg/kg to 1000 mg/kg body weight. In another embodiment, the effective amount is less than about 1, 1.5, 2, 2.5, 3, 3.5, 4, 4.5 or 5 mg/kg. In another embodiment, the effective amount is less than about 500 mg/kg, about 400 mg/kg, about 300 mg/kg, about 200 mg/kg, about 100 mg/kg, about 50 mg/kg, about 25 mg/kg, about 10 mg/kg, about 1 mg/kg, about 0.5 mg/kg, about 0.25 mg/kg, about 0.1 mg/kg, about 100 μg/kg, about 75 μg/kg, about 50 μg/kg, about 25 μg/kg, about 10 μg/kg, or about 1 μg/kg. However, the effective amount of the compound can be determined by the attending physician and depends on the condition treated, the compound administered, the route of delivery, age, weight, severity of the patient's symptoms and response pattern of the patient.

Toxicity and therapeutic efficacy of the compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue, e.g., bone or cartilage, in order to minimize potential damage to uninfected cells and, thereby, reduce side effects.

The data obtained from cell culture assays (such as those described in the examples below) and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

One or more of the compounds discussed herein may be administered in combination with other pharmaceutical agents, as well as in combination with each other. The term “pharmaceutical” agent as used herein refers to a chemical compound which results in a pharmacological effect in a patient. A “pharmaceutical” agent can include any biological agent, chemical agent, or applied technology which results in a pharmacological effect in the subject.

In addition to the components described above, the compositions may contain one or more medications or therapeutic agents which are used to treat solid tumors. In one embodiment, the additional therapy is selected from a chemotherapeutic agent, which includes, adjuvant therapy, immunotherapy or targeted therapy, surgical removal of a malignancy, and radiation therapy.

In one embodiment, the medication is a chemotherapeutic. Examples of chemotherapeutics include those recited in the “Physician's Desk Reference”, 64^{th} Edition, Thomson Reuters, 2010, which is hereby incorporated by reference. Therapeutically effective amounts of the additional medication(s) or therapeutic agents are well known to those skilled in the art. However, it is well within the attending physician to determine the amount of other medication to be delivered.

In one embodiment, the chemotherapeutic is imiquimod. In one embodiment, the chemotherapeutic is trametinib (Mekinist). In one embodiment, the chemotherapeutic is cobimetinib. In one embodiment, the chemotherapeutic is imatinib (Gleevec). In one embodiment, the chemotherapeutic is nilotinib. In one embodiment, the chemotherapeutic is nivolumab [Opdivo]. In one embodiment, the chemotherapeutic is ipilimumab [Yervoy]. In another embodiment, the additional agent is interferon.

In one embodiment, the chemotherapeutic is selected from among cisplatin, carboplatin, 5-fluorouracil, cyclophosphamide, oncovin, vincristine, prednisone, or rituximab, mechlorethamine, cyclophosphamide, ifosfamide, melphalan, chlorambucil, carmustine, lomustine, semustine, thriethylenemelamine, triethylene thiophosphoramide, hexamethylmelamine altretamine, busulfan, triazines dacarbazine, methotrexate, trimetrexate, fluorodeoxyuridine, gemcitabine, cytosine arabinoside, 5-azacytidine, 2,2′-difluorodeoxycytidine, 6-mercaptopurine, 6-thioguanine, azathioprine, 2′-deoxycoformycin, erythrohydroxynonyladenine, fludarabine phosphate, 2-chlorodeoxyadenosine, camptothecin, topotecan, irinotecan, paclitaxel, vinblastine, vincristine, vinorelbine, docetaxel, estramustine, estramustine phosphate, etoposide, teniposide, mitoxantrone, mitotane, or aminoglutethimide.

In one embodiment, the compound is combined with one or more of these pharmaceutical agents, i.e., delivered to the patient concurrently. In another embodiment, the compound is administered to the patient concurrently therewith one or more of these pharmaceutical agents. In a further embodiment, the compound is administered prior to one or more of these pharmaceutical agents. In still another embodiment, the compound is administered subsequent to one or more of these pharmaceutical agents.

These pharmaceutical agents may be selected by one of skilled in the art and thereby utilized in combination with a telomerase inhibitor and an agent that decreases or downregulates anti-oxidant response or induces oxidative stress. Examples of these additional agents include, without limitation, cytokines (interferon (α, β, γ) and interleukin-2), lymphokines, growth factors, antibiotics, bacteriostatics, enzymes (L-asparaginase), biological response modifiers (interferon-alpha; IL-2; G-CSF; and GM-CSF), differentiation agents (retinoic acid derivatives), radiosensitizers (metronidazole, misonidazole, desmethylmisonidazole, pimonidazole, etanidazole, nimorazole, RSU 1069, E09, RB 6145, SR4233, nicotinamide, 5-bromodeozyuridine, 5-iododeoxyuridine, bromodeoxycytidine), hormones (adrenocorticosteroids, prednisone, dexamethasone, aminoglutethimide), progestins (hydroxyprogesterone caproate, medroxyprogesterone acetate, megestrol acetate), estrogens (diethylstilbestrol, ethynyl estradiol/equivalents), antiestrogens (tamoxifen), androgens (testosterone propionate, fluoxymesterone), antiandrogens (flutamide, gonadotropin-releasing hormone analogs, leuprolide), photosensitizers (hematoporphyrin derivatives, Photofrin®, benzoporphyrin derivatives, Npe6, tin etioporphyrin, pheoboride-α, bacteriochlorophyll-α, naphthalocyanines, phthalocyanines, and zinc phthalocyanines), proteosome inhibitors (bortezomib), tyrosine kinase inhibitors (imatinib mesylate, dasatinib, nilotinib, MK-0457, and Omacetaxine), immunotherapeutics, vaccines, or biologically active agents.

### III. METHODS

One aspect of the invention provides a method of treating cancer in a subject in need thereof. This aspect is based on the inventor's discovery that the combination of telomerase inhibitors with an agent that decreases or downregulates anti-oxidant response or induces oxidative stress triggered redox imbalance, enhanced suppression of NRAS mutant melanoma, and significantly prolonged mouse survival.

In one embodiment, the method of treating cancer in a subject in need thereof includes administering a pharmaceutical composition or combination comprising a telomerase inhibitor and an agent that decreases or downregulates anti-oxidant response or induces oxidative stress. The pharmaceutical composition or combination may be any composition as described herein.

In one embodiment, the cancer being treated is any of those described herein or which may be benefitted by the treatment of a telomerase inhibitor and an agent that decreases or downregulates anti-oxidant response or induces oxidative stress co-therapy. In one embodiment, the method includes the treatment of cancer and tumor cells selected from, but not limited to, breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, acute and chronic lymphocytic and myelocytic leukemia, myeloma, Hodgkin's and non-Hodgkin's lymphoma, and multidrug resistant cancer. In one embodiment, the cancer is a drug resistant cancer. In one embodiment, the cancer is a melanoma. In another embodiment, the cancer is a NRAS mutant cancer. Such NRAS mutant cancers include, without limitation, somatic rectal cancer, follicular thyroid cancer, autoimmune lymphoproliferative syndrome, Noonan syndrome, juvenile myelomonocytic leukemia, giant congenital melanocytic nevus, core binding factor acute myeloid leukemia, cytogenetically normal acute myeloid leukemia, lung cancer, and melanoma. Other NRAS mutant cancers include melanoma, thyroid, endometrial, non-small cell lung, ovarian and colorectal cancers. See, e.g., Madhusudanannair et al, Journal of Clinical Oncology 30, no. 15_suppl (May 20, 2012) 3106-3106, which is incorporated herein by reference. In another embodiment, the cancer is NRAS mutant melanoma. In another embodiment, the cancer is NRAS mutant brain cancer. In another embodiment, the cancer is NRAS mutant neuroblastoma.

The therapeutic compositions administered in the performance of these methods, e.g., a telomerase inhibitor and an agent that decreases or downregulates anti-oxidant response or induces oxidative stress, may be administered directly into the environment of the targeted cell undergoing unwanted proliferation, e.g., a cancer cell or targeted cell (tumor) microenvironment of the patient. In an alternative embodiment, the compositions are administered systemically, without regard to the location of the cancer, i.e., parenteral administration. Conventional and pharmaceutically acceptable routes of administration include, but are not limited to, systemic routes, such as intraperitoneal, intravenous, intranasal, intravenous, intramuscular, intratracheal, subcutaneous, and other parenteral routes of administration or intratumoral or intranodal administration, as discussed above.

Routes of administration may be combined, if desired. In some embodiments, the administration is repeated periodically. Dosages may be administered continuously for a certain period of time, or periodically every week, month, or quarter, dependent on the condition and response of the patient, as determined by a physician. In one embodiment, the compositions i.e., a telomerase inhibitor and an agent that decreases or downregulates anti-oxidant response or induces oxidative stress, are administered at the same time. In another embodiment, the compositions are administered sequentially. In another embodiment, telomerase inhibitor is administered first. In another embodiment, the agent that decreases or downregulates anti-oxidant response or induces oxidative stress is administered first. In another embodiment, the compositions are administered within a suitable period, e.g., hours, days or weeks of each other. These periods may be determined by a physician. In one embodiment, the compositions are administered periodically, e.g. every day, week, two weeks, monthly, quarterly, or as prescribed by physician.

These therapeutic compositions may be administered to a patient preferably suspended in a biologically compatible solution or pharmaceutically acceptable delivery vehicle, as discussed herein. The various components of the compositions are prepared for administration by being suspended or dissolved in a pharmaceutically or physiologically acceptable carrier such as isotonic saline; isotonic salts solution or other formulations that will be apparent to those skilled in such administration. The appropriate carrier will be evident to those skilled in the art and will depend in large part upon the route of administration. Other aqueous and non-aqueous isotonic sterile injection solutions and aqueous and non-aqueous sterile suspensions known to be pharmaceutically acceptable carriers and well known to those of skill in the art may be employed for this purpose.

In one embodiment, the methods described herein include administration of a telomerase inhibitor and an agent that decreases or downregulates anti-oxidant response or induces oxidative stress, as described above, in combination with other known anti-proliferative disease therapies. In one embodiment, the additional therapy is selected from a chemotherapeutic agent (adjuvant therapy, immunotherapy or targeted therapy), surgical removal of a malignancy, and radiation therapy, as described above.

In one embodiment of such combination therapy, the present method can include administration of a passive therapeutic that can immediately start eliminating the targeted cell undergoing unrestricted or abnormal replication or proliferation, e.g., tumor. This can also be accompanied by administration of active immunotherapy to induce an active endogenous response to continue the tumor eradication. Such immune-based therapies can eradicate residual disease and activate endogenous antitumor responses that persist in the memory compartment to prevent metastatic lesions and to control recurrences. This treatment may occur, before, during or after administration of the telomerase inhibitor and/or the agent that decreases or downregulates anti-oxidant response or induces oxidative stress. In another example, surgical debulking, in certain embodiments is a necessary procedure for the removal of large benign or malignant masses, and can be employed before, during or after application of the methods and compositions as described herein. Chemotherapy and radiation therapy, in other embodiments, bolster the effects of the methods described herein. Such combination approaches (surgery plus chemotherapy/radiation plus immunotherapy) with the methods of administering a telomerase inhibitor and an agent that decreases or downregulates anti-oxidant response or induces oxidative stress are anticipated to be successful in the treatment of many proliferative diseases.

Still other adjunctive therapies for use with the methods and compositions described herein include non-chemical therapies. In one embodiment, the adjunctive therapy includes, without limitation, acupuncture, surgery, chiropractic care, passive or active immunotherapy, X-ray therapy, ultrasound, diagnostic measurements, e.g., blood testing. In one embodiment, these therapies are to be utilized to treat the patient. In another embodiment, these therapies are utilized to determine or monitor the progress of the disease, the course or status of the disease, relapse or any need for booster administrations of the compounds discussed herein.

### IV. KITS

Also provided herein are kits or packages of compositions containing a telomerase inhibitor and an agent that decreases or downregulates anti-oxidant response or induces oxidative stress. The kits may be organized to indicate a single formulation or combination of formulations to be taken at each desired time.

Suitably, the kit contains packaging or a container with a telomerase inhibitor and an agent that decreases or downregulates anti-oxidant response or induces oxidative stress formulated for the desired delivery route. In one embodiment, the kit contains instructions on dosing and an insert regarding the active agent(s). In another embodiment, the kit may further contain instructions for monitoring circulating levels of the components of the composition and materials for performing such assays including, e.g., reagents, well plates, containers, markers or labels, and the like. Such kits are readily packaged in a manner suitable for treatment of a desired indication. Other components for inclusion in the kits will be readily apparent to one of skill in the art, taking into consideration the desired indication and the delivery route.

The compositions described herein can be a single dose or for continuous or periodic discontinuous administration. For continuous administration, a package or kit can include a telomerase inhibitor and an agent that decreases or downregulates anti-oxidant response or induces oxidative stress in each dosage unit (e.g., solution, lotion, tablet, pill, or other unit described above or utilized in drug delivery), or separate dosage units for each component, and optionally instructions for administering the doses daily, weekly, or monthly, for a predetermined length of time or as prescribed. When the composition is to be delivered periodically in a discontinuous fashion, a package or kit can include placebos during periods when the composition is not delivered. When varying concentrations of the composition, the components of the composition, or the relative ratios of a telomerase inhibitor and an agent that decreases or downregulates anti-oxidant response or induces oxidative stress within the composition over time is desired, a package or kit may contain a sequence of dosage units which provide the desired variability.

A number of packages or kits are known in the art for dispensing the compositions for periodic oral use. In one embodiment, the package has indicators for each period. In another embodiment, the package is a labeled blister package, dial dispenser package, or bottle. The composition may also be sub-divided to contain appropriate quantities of a telomerase inhibitor and an agent that decreases or downregulates anti-oxidant response or induces oxidative stress. For example, the unit dosage may be packaged compositions, e.g., packeted powders, vials, ampoules, prefilled syringes or sachets containing liquids.

The packaging means of a kit may itself be geared for administration, such as an inhalant, syringe, pipette, eye dropper, or other such apparatus, from which the formulation may be applied to an affected area of the body, such as the lungs, injected into a subject, or even applied to and mixed with the other components of the kit.

The compositions also may be provided in dried or lyophilized forms. When reagents or components are provided as a dried form, reconstitution generally is by the addition of a suitable solvent. Such formulations can be stored either in a ready-to-use form or in a form requiring reconstitution prior to administration. The formulations may also be contained with a container having a sterile access port, for example, an intravenous solution bag or vial having a stopper pierceable by a hypodermic injection needle. It is envisioned that the solvent also may be provided in another package.

The kits also may include a means for containing the vials in close confinement for commercial sale such as, e.g., injection or blow-molded plastic containers into which the desired vials are retained. The kits may further include, or be packaged with a separate instrument for assisting with the injection/administration or placement of the composition within the body of an animal. Such an instrument may be an inhaler, syringe, pipette, forceps, measuring spoon, eye dropper or any such medically approved delivery means.

In one embodiment, a kit is provided and contains a telomerase inhibitor and an agent that decreases or downregulates anti-oxidant response or induces oxidative stress. These components may be in the presence or absence of one or more of the carriers or excipients described above. The kit may optionally contain instructions for administering the composition to a subject.

In a further embodiment, a kit is provided and contains a telomerase inhibitor in a first dosage unit, an agent that decreases or downregulates anti-oxidant response or induces oxidative stress in a second dosage unit, and one or more of the carriers or excipients described above in a third dosage unit. The kit may optionally contain instructions for administration.

It should be understood that the embodiments described herein (e.g., Sections I Definitions, II Compositions, III Methods and IV Kits) are intended to be applied to other compositions, regiments, aspects, embodiments, methods and kits described across the Specification.

### IV. EXAMPLES

The invention is now described with reference to the following examples. These examples are provided for the purpose of illustration only. The compositions, experimental protocols and methods disclosed and/or claimed herein can be made and executed without undue experimentation in light of the present disclosure. The protocols and methods described in the examples are not considered to be limitations on the scope of the claimed invention. Rather this specification should be construed to encompass any and all variations that become evident as a result of the teaching provided herein. One of skill in the art will understand that changes or variations can be made in the disclosed embodiments of the examples, and expected similar results can be obtained. For example, the substitution of reagents that are chemically or physiologically related for the reagents described herein are anticipated to produce the same or similar results. All such similar substitutes and modifications are apparent to those skilled in the art and fall within the scope of the invention.

Targeting RAS is one of the greatest challenges in cancer therapy. Oncogenic mutations in NRAS are present in over 25% of melanomas patients whose tumors harbor NRAS mutations have limited therapeutic options and poor prognosis. Thus far, there are no clinical agents available to effectively target NRAS or any other RAS oncogene. An alternative approach is to identify and target critical tumor vulnerabilities or non-oncogene addictions that are essential for tumor survival. We investigated the consequences of NRAS blockade in NRAS mutant melanoma and show that decreased expression of the telomerase catalytic subunit, TERT, is a major consequence. TERT silencing or treatment of NRAS mutant melanoma cells with the telomerase-dependent telomere uncapping agent 6-thio-2′-deoxy guanosine (6-thio-dG), led to rapid cell death of NRAS mutant melanoma, along with evidence of both telomeric and non-telomeric DNA damage, increased ROS levels, and upregulation of a mitochondrial anti-oxidant adaptive response. Combining 6-thio-dG with the mitochondrial inhibitor Gamitrinib attenuated this adaptive response and more effectively suppressed NRAS-mutant melanoma. Described herein is a robust dependency of NRAS mutant melanoma on TERT, and proof of principle for a new combination strategy to combat this class of tumors, which could be expanded to other tumor types.

Despite the critical role of telomerase in cancer, developing effective anti-telomerase therapies has been challenging (Jafri et al., 2016; Shay, 2016). Only one compound, imetelstat (GRN163L), an oligonucleotide that binds to the RNA subunit of Telomerase TERC, has shown efficacy in myeloproliferative disorders, but has displayed limited activity in solid tumors (Baerlocher et al., 2015; Jafri et al., 2016; Tefferi et al., 2015). Preclinical approaches to inhibit telomerase have been evaluated in melanoma; nevertheless, telomerase inhibitors have shown modest activity as monotherapy and they have not yet been successfully translated to the clinical setting (Bagheri et al., 2006; Nosrati et al., 2004; Puri et al., 2004). Interestingly, inactivation of p2lcombined with treatment with imetelstat and CP-31398 (to restore p53 activity), repressed melanoma growth (Gupta et al., 2014), suggesting that telomerase-based combination approaches might lead to enhanced anti-tumor effects.

The lack of success developing effective anti-telomerase approaches and the reason why solid tumors respond poorly to telomerase monotherapy remains elusive. This could be in part related to early efforts focusing mainly on targeting the reverse transcriptase activity of telomerase and being heavily reliant on telomere shortening. A shortcoming of this approach is the relatively long lag period needed for efficacy, which could trigger activation of adaptive mechanisms and drug resistance. In our studies, we found that either TERT silencing or treatment with 6-thio-dG led to telomere dysfunction and relatively rapid cell death. Even though we did not detect significant changes in average telomere length within the time frame that cells underwent apoptosis, it is possible that critically short telomeres, commonly present in melanoma cells, become uncapped and dysfunctional, thereby also contributing to cell death. Of note, it has been reported that melanoma cells harbor a subset of critically short telomeres and that the upregulation of telomerase activity associated with TERT promoter mutations does not preclude telomere attrition. In fact, Chiba and colleagues recently demonstrated that reactivation of telomerase via TERT promoter mutations allows for melanomagenesis first by protecting the shortest telomeres rather than by elongating the telomeres, and subsequently by sustaining tumor cell proliferation. These studies and our results suggest that anti-melanoma strategies dependent solely on telomere shortening may not have a significant impact on tumor maintenance. In contrast, induction of telomere uncapping and dysfunction appears to have a more rapid effect triggering apoptosis in a telomere length independent manner. In support of this premise, Blasco and colleagues recently showed that induction of acute telomere uncapping by inhibition of the shelterin protein TRF1, can restrain tumor growth independently of telomere length in a p53-null K-Ras (G12V)-induced lung carcinoma mouse model Likewise, it has been shown that ectopic expression of telomerase can protect cells against double-strand DNA damage in a telomere lengthening independent manner ¹¹. We found that non-toxic doses of the telomere uncapping agent 6-thio-dG as a single agent slowed, but did not completely abrogated tumor growth in vivo, raising the possibility that induction of telomere dysfunction could prompt the activation of adaptive or compensatory survival mechanisms mitigating the effects of the drug.

While resistance to TERT-based approaches has been attributed primarily to engagement of alternative lengthening of telomeres (ALT) (Hu et al., 2012; Queisser et al., 2013), little is known about adaptive survival mechanisms triggered by TERT inhibition or telomere dysfunction. We found that whereas telomere dysfunction is associated with oxidative damage, melanoma cells rapidly activate an anti-oxidant response coupled to increased expression of PGC1-a and ROS scavenging enzymes, primarily Mn-superoxide dismutase SOD2, with no evidence of ALT activation. These results led us to postulate that a rational strategy could involve combining 6-Thio-dG with agents that further induce oxidative stress and disable the anti-oxidant machinery of tumor cells. We selected Gamitrinib, a mitochondriotoxic small molecule that selectively blocks mitochondrial HSP90 and exhibits broad anti-cancer activity including efficacy in BRAF-mutant melanoma, but limited activity in NRAS mutant cells as a single agent (Chae et al., 2012). Notably, several studies have reported that melanoma drug resistance can be mediated by mitochondrial adaptive responses (Haq et al., 2013; Roesch et al., 2013; Zhang et al., 2016). Likewise, Hu et al, demonstrated in an ATM deficient lymphoma mouse model that genetic ablation of telomerase caused cell death, but also induced ALT and PGC-1ß (Hu et al., 2012). They further showed that genetic depletion of PGC-1ß impaired mitochondria function, enhancing anti-telomerase therapy. Altogether these studies suggest that mitochondrial function might play a major role modulating multi-drug response and cancer cell viability.

The exact mechanism triggering the mitochondrial detoxifying response prompted by TERT depletion or treatment with 6-Thio-dG needs to be further investigated. Several lines of evidence suggest that TERT plays other roles independent of its canonical telomere lengthening function (Li and Tergaonkar, 2014). For example, TERT has been shown to traffic to mitochondria; however, the relevance of mitochondrial TERT is not fully understood. Previous reports suggest that ROS can modulate TERT intracellular localization and that TERT can bind to mitochondrial DNA, promoting resistance to oxidative stress and increasing cell survival (Sharma et al., 2012). Consistent with our studies, TERT overexpression attenuates ROS basal levels and diminishes stressed-induced ROS generation (Indran et al., 2011). We found that both TERT depletion and treatment with 6-thio-dG induced generation of ROS and that TERT can potentiate the antioxidant capacity of melanoma cells in a RT and telomere-lengthening independent manner, enabling melanoma cells to survive under conditions of excessive oxidative stress. Since ROS signaling can induce the expression of FOXO transcription factors, FOXO along with the transcriptional co-activator PGC-1α could enhance the expression of detoxifying enzymes genes such as SOD2 and catalase in NRAS mutant melanoma cells (Kops et al., 2002; Olmos et al., 2009).

Our studies support the notion that melanomas are highly dependent on TERT for survival and that NRAS mutant melanoma is a prime candidate for TERT-based therapeutic approaches. Our data suggest that in addition to a telomere- dependent role of TERT in melanoma, TERT may also possess a telomere lengthening-independent role promoting melanoma survival, as catalytically impaired and telomere elongating deficient TERT can protect cells from loss of oncogenic NRAS. These results could have therapeutic implications, as they raise the possibility that approaches that solely target the reverse transcriptase activity of TERT could have limited efficacy impairing tumor growth and maintenance. Altogether, our studies stress the need to develop drug combinations co-targeting not only telomerase's catalytic activity but also other telomere-lengthening independent functions and adaptive resistance mechanisms. Our data provide proof-of-principle for this strategy and for developing similar combinations to increase anti-tumor responses. Finally, it would be important in future studies to determine whether this approach could also be applied to other melanoma subtypes and other RAS-driven tumors.

### Example 1

### Materials and Methods

### Cell Culture, Viability and Cell Death Assays

Human patient-derived melanoma cell lines were cultured in RPMI-1640 medium supplemented with 5% fetal bovine serum and grown at 37° C. in 5% CO₂. All cell lines were periodically authenticated by DNA finger printing using Life Technologies AmpFISTR Identifier microsatellite kit and tested for mycoplasma by Lonza Mycoalert Assay. NRAS and BRAF-V600E mutation status was verified by direct Sanger sequencing.

Cells were seeded in 96-well plates and treated with drugs. After 6 days, cell viability was assessed following 6h incubation with 500 μM Alamar Blue (ThermoFisherScientific, Philadelphia, Pa.) by measuring fluorescence using an EnVision Xcite Multilabel plate reader (Perkin Elmer, Waltham, Mass.). Cell death was determined by flow cytometry using PSVue-643 (Molecular Targeting Technologies) or Annexin V (640919, Biolegend, San Diego, Calif.) and Propidium Iodide (Sigma) staining. Samples were analyzed using a BD LSRII flow cytometer (BD Biosciences, San Jose, Calif.).

### TERT Constructs, Small Hairpin RNA, and Lentivirus Infection

Lentiviral NRAS shRNA in pLKO1 backbone and TERT shRNA in pGIPZ backbone were obtained from Thermo Scientific (Waltham, MA). TERT constructs (Wild-type, FVYL1016 or FVYL1028) in a pBlast lentiviral vector have been previously described (52). Lentiviruses were produced by transfection of 293T cells with packaging plasmids (pPAX2 and pMD2.G) along with 4 μg lentiviral shRNA vector using Lipofectamine 2000 reagent (Invitrogen, Waltham, Mass.) following the manufacturer's instructions. Melanoma cells were transduced with virus in the presence of 6 μg/mL polybrene (Sigma-Aldrich, St.Louis, Mo.) for 18 h. Transduced cell populations were selected with appropriate antibiotics. shRNA knockdown efficiency was determined by western blot analysis and/or qRT-PCR.

### PCR Array

Human cellular senescence RT2 Profiler PCR array (Qiagen, Valencia, Calif.) was used following manufacturer's specifications. Data was analyzed with the SABiosciences PCR Array Data Analysis Template Excel.

Quantitative Real Time PCR (qRT-PCR)

Total RNA (1 μg) was reverse transcribed using Maxima First-Strand cDNA synthesis kit (Thermo Scientific, Waltham, Mass.). Fast SYBR Green Master Mix (Applied Biosystems, Waltham, Mass.) was used with 100 ng cDNA template and 250 nM primers for the evaluation of target gene expression. Primers sequences are listed in Table SI. Amplification was performed in triplicate using ABI PRISM 7500 Fast RT PCR System (Applied Biosystems, Waltham, Mass.) and expression of mRNA was assessed using the aCt method.

### Immunobloting

For western blot analysis, cells were washed with cold PBS containing 100 μM Na₃VO₄, scraped, collected by centrifugation, and quick-frozen in dry ice before lysis. Cells were lysed with THE buffer (50 mM Tris-HCl, 2 mM EDTA, 25 mM NaCl, 1% NP40 and protease inhibitors) and equal amounts of protein (30-50 μg) were subjected to SDS-PAGE and proteins transferred onto nitrocellulose membranes (Bio-Rad). Nitrocellulose membranes were incubated overnight with primary antibodies at 4° C., followed by lh incubation with Alexa Fluor-labeled secondary antibodies (IRDye 680LT goat-anti mouse or IRDye 800CW goat-anti rabbit antibodies (LI-COR Biosciences) at room temperature. Fluorescent images were acquired and quantified by LI-COR Odyssey Imaging System. Vinculin and Actin antibodies (Sigma-Aldrich, St. Louis, Mo.) were used as loading controls. NRAS (SC-519) from Santa Cruz Biotechnology (Dallas, Tex.) and TERT (600-401-252) from Rockland (Limerick, Pa.) and SOD2 (13141) from Cell Signaling (Danvers, Mass.).

### Telomerase Activity Assay

Telomerase activity was measured by TRAP assay as previously described (Mender et al., 2015). Briefly, cells were lysed with NP-40 buffer (10 mM Tris-HCl, 1 mM MgCl₂, 1 mM EDTA, 1% NP-40, 0.25 mM sodium deoxycholate, 10% glycerol, 150 mM NaCl, 5 mM β-Mercaptoethanol) for 30 minutes. HCT116 cells and lysis buffer were used as positive and negative controls respectively. Telomerase extension products were amplified by PCR and run on 10% non-denaturing acrylamide gel. Typhoon Phosphoimager scanner (Molecular Dynamics, GE Healthcare, Little Chalfont, UK) was used for visualization of gel products.

### Telomere Length Assay (TRF)

Genomic DNA was prepared using Wizard genomic DNA purification kit (Promega) following manufacturer's instruction. For telomere length and Southern blot analysis, genomic DNA (˜5 μg) was digested with AluI+MboI restriction endonucleases, fractionated in a 0.7% agarose gel, denatured, and transferred onto a GeneScreen Plus hybridization membrane (PerkinElmer). The membrane was cross-linked, hybridized overnight at 42° C. with 5′-end-labeled ³²P-(TTAGGG)₄ probe in Church buffer (0.5 N Na₂HPO4 pH 7.2, 7% SDS, 1% BSA, 1 mM EDTA), and washed twice for 5 min each with 0.2 N wash buffer (0.2 N Na₂HPO4 pH 7.2, 1 mM EDTA, and 2% SDS) at room temperature and once for 10 min with 0.1 N wash buffer at 42° C. The images were analyzed with Phosphorimager, visualized by Typhoon 9410 Imager (GE Healthcare), and processed with ImageQuant 5.2 software (Molecular Dynamics).

### DNA Damage and Telomere Dysfunction Assay (TIF)

For assessment of global DNA damage cells were fixed and permeabilized with BD CytoFix/Perm reagent following the manufacturer's instructions and incubated with γH2AX antibody (Cell Signaling Technology, Danvers, Mass.) for 1 h at room temperature followed by 1 h incubation with Alexa-647 anti-Rabbit secondary antibody. Mean fluorescence staining was quantified by flow cytometry. For NAC treatment cells were treated with 1 mM N-Acetyl-N-cysteine (Sigma Aldrich) in PBS for the duration of the experiment. NAC was replaced every 48 h up to day seven.

For TIF analysis, indirect immunofluorescence (IF) combined with fluorescence in situ hybridization (FISH) was performed as previously described with some modifications. Briefly, 1×105 cells grown on coverslips were fixed for 15 min in 1% paraformaldehyde in PBS at RT, followed by permeabilization for 15 min in 1× PBS/0.3% Triton X-100 at RT. After washing with 1× PBS, cells were incubated for 60 min in PBG blocking solution (0.5% BSA, 0.2% fish gelatin in 1× PBS) before immuno-staining. Primary antibodies were prepared in blocking solution with following dilutions: anti-53BP1 (1:500, IHC-00001; Bethyl Laboratories), and anti-[Symbol]H2AX (1:500, 05-636; Millipore). After IF, cells were fixed in 4% paraformaldehyde in 1× PBS for 10 min and dehydrated in ethanol series (70%, 95%, 100%). Coverslips were incubated 3 min at 80-85[Symbol]C in hybridization mix [70% formamide, 10 mM Tris-HCl, pH 7.2, and 0.5% blocking solution (Roche)] containing telomeric PNA-Tamra-(CCCTAA)3 probe, followed by hybridization at room temperature in dark moisturized chambers for 2 hrs. Coverslips were washed twice for 15 min each with 70% formamide, 10 mM Tris-HCl (pH 7.2), and 0.1% BSA, followed by three 5 min washes with 0.15 M NaCl, 0.1 M Tris-HCl (pH 7.2), and 0.08% Tween-20. Nuclei were counterstained with 0.1 [Symbol]g/ml DAPI in blocking solution and slides were mounted with Fluoromount-G (0100-01, SouthernBiotech). Images were captured with a 100× objective on a Nikon E600 Upright microscope (Nikon Instruments, Inc., Melville, N.Y.) using ImagePro Plus software (Media Cybernetics, Silver Spring, Md.) for image processing. Cells with >4 53BP1 or γH2AX foci colocalizing with telomere DNA foci were scored as TIF positive. TIF were quantified from at least three independent experiments.

### Measurement of Reactive Oxygen Species

General or mitochondrial specific ROS were measured by flow cytometry H2DCFDA or MitoSoxRed (Invitrogen, Waltham, Mass.) following manufacturer's specifications.

### 3D Tumor Spheroid Models

5000 cells were seeded in 96-well plates coated with 1% agar in PBS and allowed to grow for 72 h. Spheroids were embedded into rat collagen type I (Corning, Bedford, Mass.) mixture as previously described (Villanueva, et al, 2010) and treated with drugs for 7 days. Spheroids were stained with Live/Dead cell assay (Invitrogen, Waltham, Mass.) and imaged using a Nikon Inverted TE2000 microscope (Melville, N.Y., USA). Images were processed and merged using Image Pro software (Media Cybernetics, Rockville, Md., USA).

### Animal Studies

All animal experiments were performed in accordance with institutional guidelines. Female and male (5-6 weeks old) NOD/LtSscidIL2Rγ-null mice (NSG) mice were injected subcutaneously with 1×10⁶ melanoma cells in a suspension of matrigel (BD Matrigel Basement Membrane Matrix, Growth Factor Reduced)/RPMI media at a ratio of 1:1. Tumor growth was measured twice weekly with digital calipers. Once tumors reached an average volume of 50-100 mm³, mice were randomized into different treatment groups. For single drug studies, 2.5 mg/Kg of 6-Thio-dG was administered (i.p, q.d.). For combination studies, same dose of 6-Thio-dG was administered alone for the first 7 days (q.d.). Mice were then treated with 12.5 mg/K of Gamitrinib (i.p, q.d.) and 6-Thio-dG (ip, q.o.d.) up to 20 days.

Tumor volume over time were used to model tumor growth rate in each treatment group. Tumor growth rates were compared between treatment groups using a linear mixed-effect model or mixed-effect spline model with the random effect at individual animal level. A p-value<0.05 was considered statistically significant.

For survival studies, treatment was discontinued after 20 days, and animals were followed-up for 10 additional days. For survival analysis, tumor volume endpoint was preset at 1800 mm³ and data represented as Kaplan-Meir curves.

TABLE I
Mutation status of TERT promoter in melanoma cell lines.

| Cell line | NRAS mutation | TERT promoter mutation |
|---|---|---|
| WM3000 | Q61R | C/T (−124, −138, −139) |
| WM3451 | Q61K | T/C, C/T (−246, −139, −138) |
| M93-047 | Q61K | — |
| WM852 | Q61R | C/C, C/T (−246, −139, −138) |
| WM1366 | Q61L | T/C, C/T (−138, −139) |
| WM3629 | G12D | T/C (−124) |
| WM4113 | Q61R | A/C (−57) |

### PCR Array

Human cellular senescence RT2 Profiler PCR array (Qiagen, Valencia, Calif.) was used following manufacturer's specifications. Briefly, lug of mRNA was used for cDNA synthesis with RT2 First Strand Kit (Applied Biosystems, Waltham, Mass.). cDNA and SYBR Green mastermix dispensed into PCR array plate and placed in the 7500 real-timer cycler (Applied Biosystems, Waltham, Mass.). Data was analyzed with the SABiosciences PCR Array Data Analysis Template Excel.

TABLE II
Primers sequences

| Gene | Forward Seq. 5′-3′ | Reverse Seq.5′-3′ |
|---|---|---|
| GAPDH | TGCCAATGATGACATCAAGAA | GGAGTGGGTGTCGCTGTTG |
| TERT | GCCGATTGTGAACATGGACTA | GCTCGTAGTTGAGCACGCTG |
| | CG | AA |
| PGC1 | CTGCTAGCAAGTTTGCCTCA | AGTGGTGCAGTGACCAATCA |
| alpha | | |
| PGC1 | CAGACAGAACGCCAAGCATC | TCGCACTCCTCAATCTCACC |
| beta | | |
| SOD2 | GGCCTACGTGAACAACCTGA | CCGTTAGGGCTGAGGTTTGT |
| GPX1 | TATCGAGAATGTGGCGTCCC | CAAACTGGTTGCACGGGAAG |
| UCP2 | CCTCTCCCAATGTTGCTCGT | GGCAAGGGAGGTCATCTGTC |

### Statistical Analysis

All experiments were performed at least three independent times unless otherwise indicated; sample size (N) is indicated in the figure legends. Data are expressed as average ±SEM unless otherwise indicated. For in vitro experiments with n=3/group, we target 80% power to test a large effect size of 3.1 at a two-sided type I error rate of 5%. Results normally distributed with equal variance between groups were analyzed by unpaired two-tailed Student's t-test. If variance was not similar between groups, student's t-test with unequal variances was applied. Asterisks denote P-value significance: *p<0.05; **p<0.01; ***p<0.005. Sample sizes, statistical tests and p values are indicated in the figure legends. All statistical analyses were calculated using Stata 14, GraphPad Prism5 software, or Microsoft Excel. For microscope images (spheroids, IF) and immunoblots representative images of three independent experiments are shown.

### Example 2

### Results

A. NRAS Mutant Melanomas are Addicted to TERT

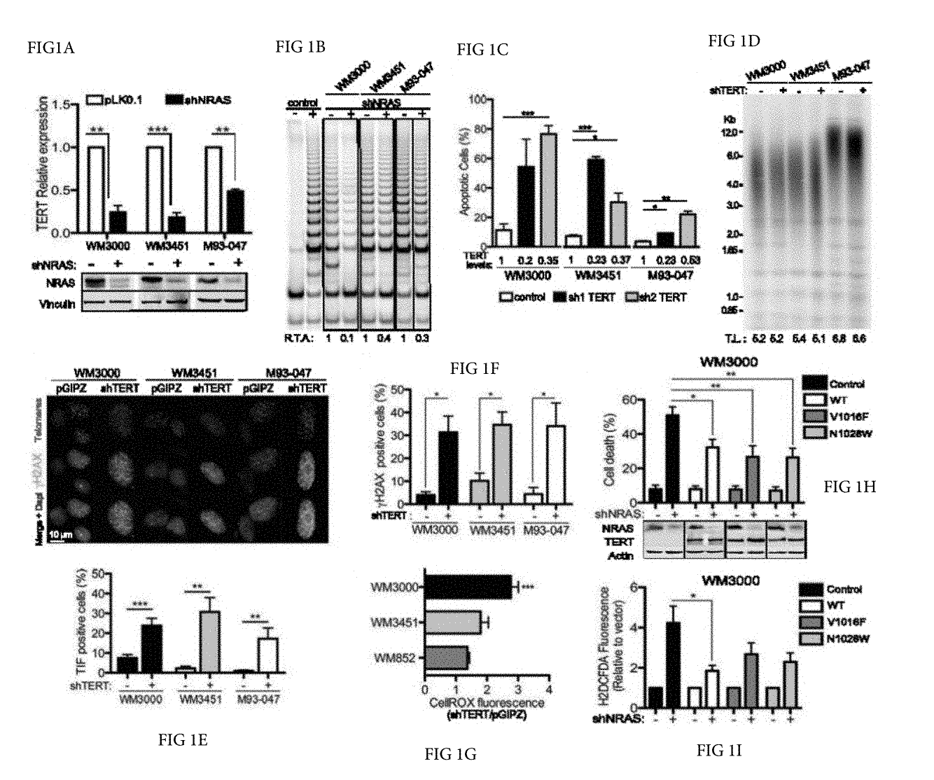

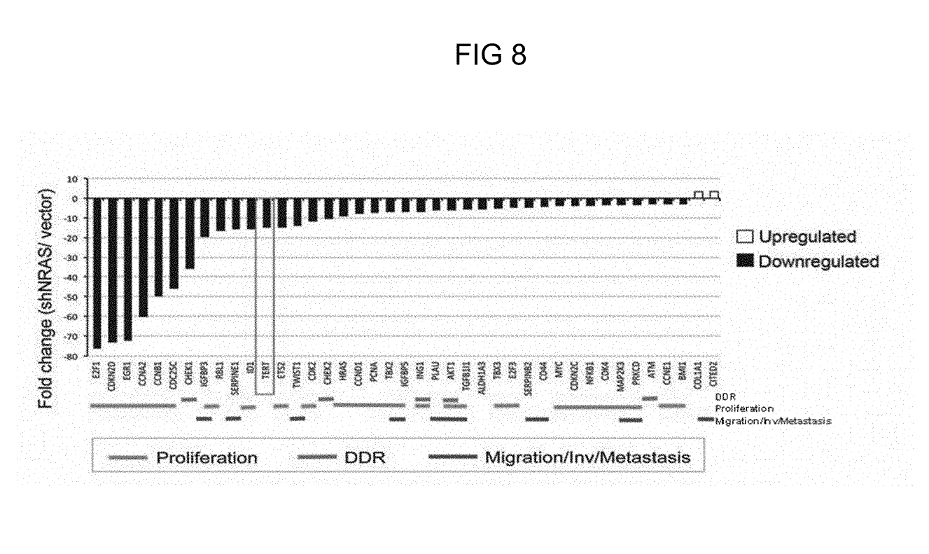

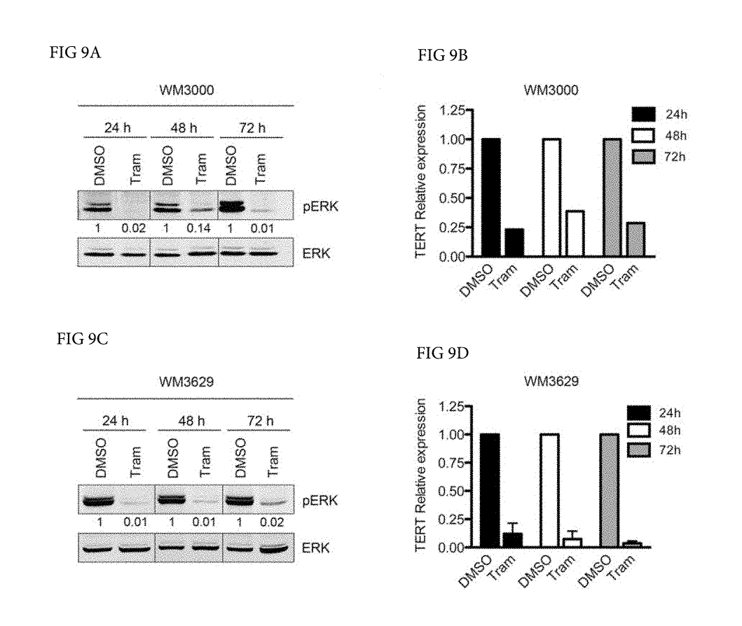

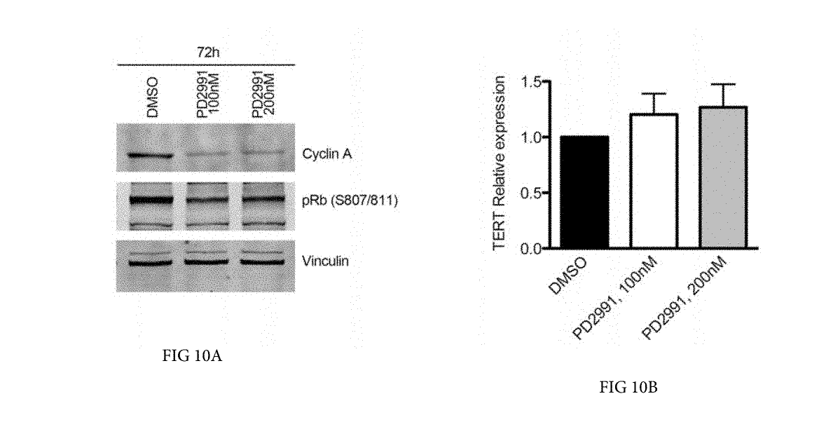

To identify specific vulnerabilities of NRAS mutant melanoma, we performed gene expression analysis in NRAS mutant melanoma cells following depletion of NRAS. We focused on genes known to regulate proliferation and senescence, as we had established that NRAS silencing rapidly triggered proliferation arrest and induced senescence. One of the most pronounced effects of NRAS silencing was downregulation of the catalytic subunit of telomerase, TERT (FIG. 1a; FIG. 8). Of note, TERT levels were downregulated following NRAS depletion in both NRAS mutant melanoma cells harboring TERT promoter mutations and to a lesser degree in melanoma cells with wild-type TERT promoter (Table 1). Downregulation of TERT was coupled to diminished telomerase activity (60-90%; FIG. 1b) Consistent with previous reports indicating that the RAS/MEK signaling pathway regulates TERT expression ^{34, 57}, treatment of NRAS mutant melanoma cells with the MEK inhibitor trametinib downregulated TERT mRNA levels (FIG. 9). Since telomerase activity is associated with cell cycle progression ¹³, we wondered if the decrease in TERT levels could be a bystander effect of cell cycle arrest elicited by NRAS silencing. To rule out this possibility, we treated NRAS mutant melanoma cells with a cdk4/6 inhibitor to induce cell cycle arrest and assessed TERT levels. Treatment with the cdk4/6 inhibitor palbociclib (PD-0332991) led to sustained proliferation arrest, but did not significantly affect TERT levels (FIG. 10).

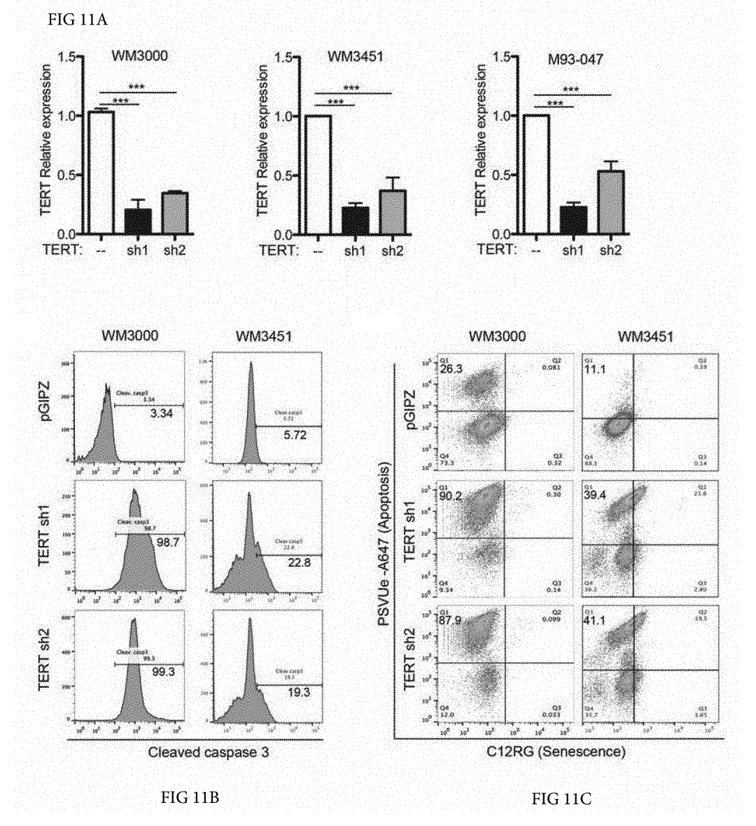

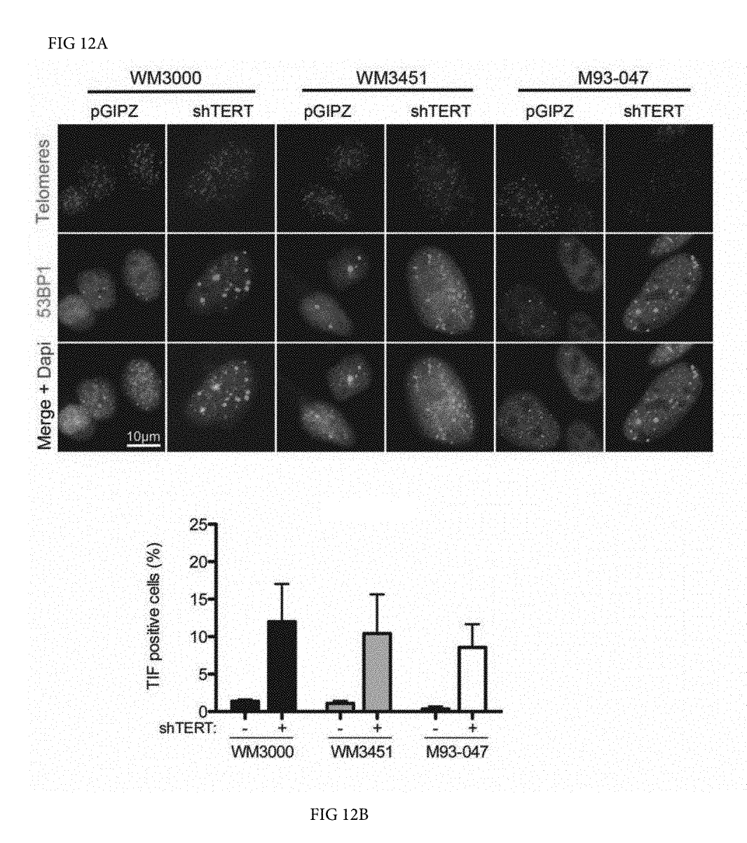

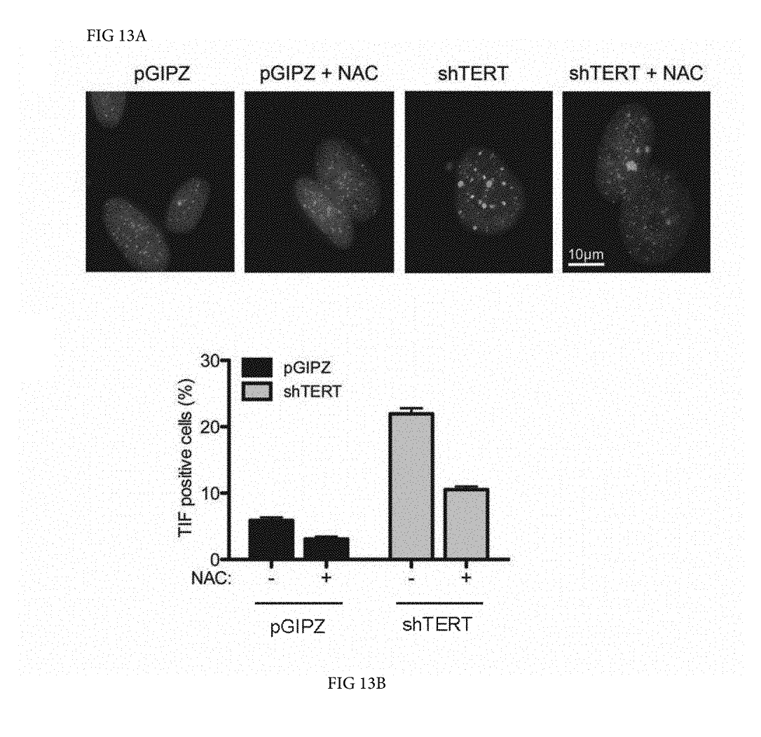

To determine the dependency of NRAS mutant melanomas on TERT, we evaluated the effect of silencing TERT in these cells (FIG. 1C). Depletion of TERT, using two different short hairpins, led to extensive and relatively rapid (7-10 days) induction of apoptosis in NRAS mutant melanoma cells, with no evidence of senescence within this time frame (FIGS. 1C and FIG. 11A-C). We next determined the effect of TERT silencing on average telomere length by southern blot of terminal restriction fragments. We selected this time frame as our data indicated that much of cell death (˜70%) occurred within 10-12 days following TERT depletion. Using this assay, we were unable to detect a significant decrease in average telomere length following transduction of shTERT (FIG. 1D), when much of cell death (˜70%) occurred. TERT depletion led to increased telomere dysfunction-induced foci (TIFs), as well as globally increased phosphorylation of histone γ-H2AXand accumulation of reactive oxygen species (FIGS. 1e-g and FIG. 12). To determine if oxidative stress was contributing to telomere dysfunction, cells transduced with non-targeting vector or TERT shRNA were treated with the antioxidant N-acetyl-L-cysteine (NAC). Treatment with NAC attenuated the number of TIF in TERT depleted cells, suggesting that oxidative stress contributes to telomeric DNA damage and dysfunction ((FIG. 13G). Together, these data support the premise that TERT depletion may lead to both telomeric and non-telomeric DNA damage in NRAS mutant melanoma cells.

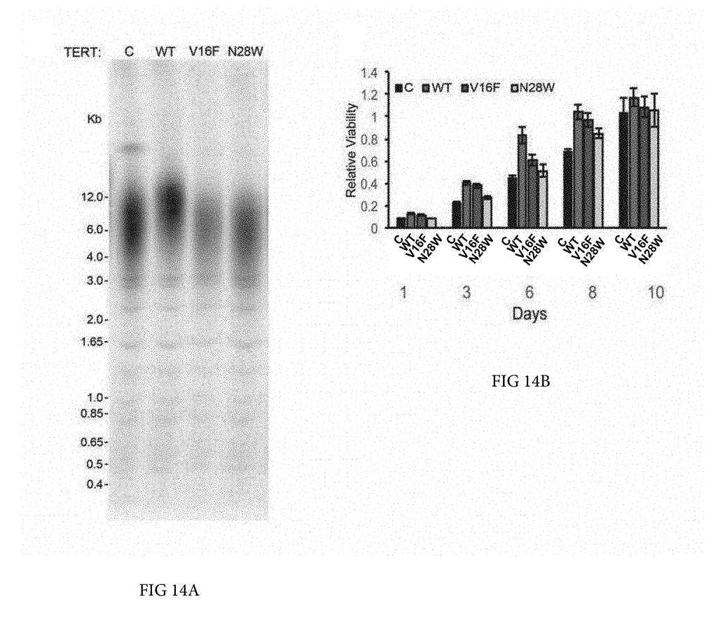

We next sought to clarify the requirement for the catalytic activity of TERT for the pro-survival function of this protein in NRAS-mutant melanoma. Toward this end, we compared the ability of ectopically expressed wild-type TERT (WT; catalytically active) with two different catalytic domain deficient constructs of TERT for their ability to rescue cell death induced by NRAS silencing. These two mutants, FVYL1016 and FVYL1028, contain point mutations in the FVYL motif, which is a conserved hydrophobic pocket on the outer surface of the TERT thumb domain (Bryan et al., 2015). Mutations of the FVYL pocket in TERT impair catalytic activity and lead to telomere attrition in primary human fibroblasts and melanoma cells ectopically expressing these constructs (FIG. 14A; (Bryan et al., 2015).

Of note, mutations within this motif do not affect proper protein folding (Bryan et al., 2015) or proliferation of NRAS mutant melanoma cells ectopically expressing these constructs (FIG. 14B) (Bryan et al., 2015). We found that ectopic expression of either wild-type (WT) TERT, or either of the two catalytically impaired TERT mutants, FVYL1016 or FVYL1028, partially protected melanoma cells from cell death induced by NRAS depletion (FIG. 1H). Similarly, ectopic expression of either WT-TERT, or to a lesser extent either of the two catalytically impaired TERT mutants could alleviate the increased levels of ROS associated with NRAS silencing (FIG. 1I). These combined data indicate that TERT contributes to survival of NRAS mutant melanoma and raises the possibility that, in addition to its catalytic role, TERT might also possess a non-catalytic survival role in melanoma.

B. The Nucleoside Analog 6-Thio-DG Induces Telomere Dysfunction and Cell Death in NRAS Mutant Melanoma

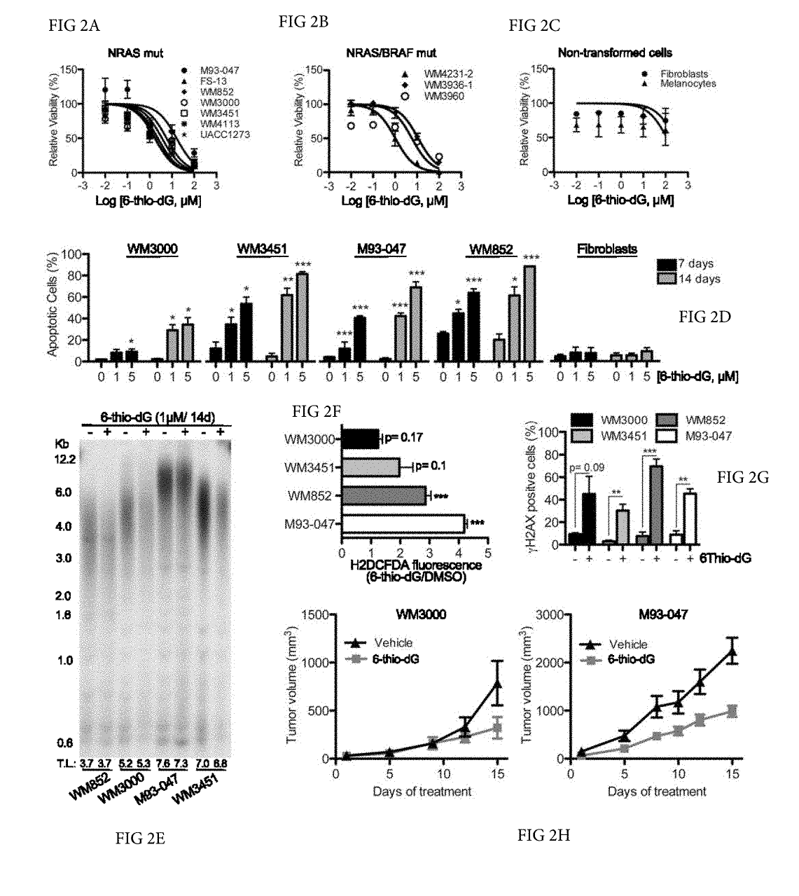

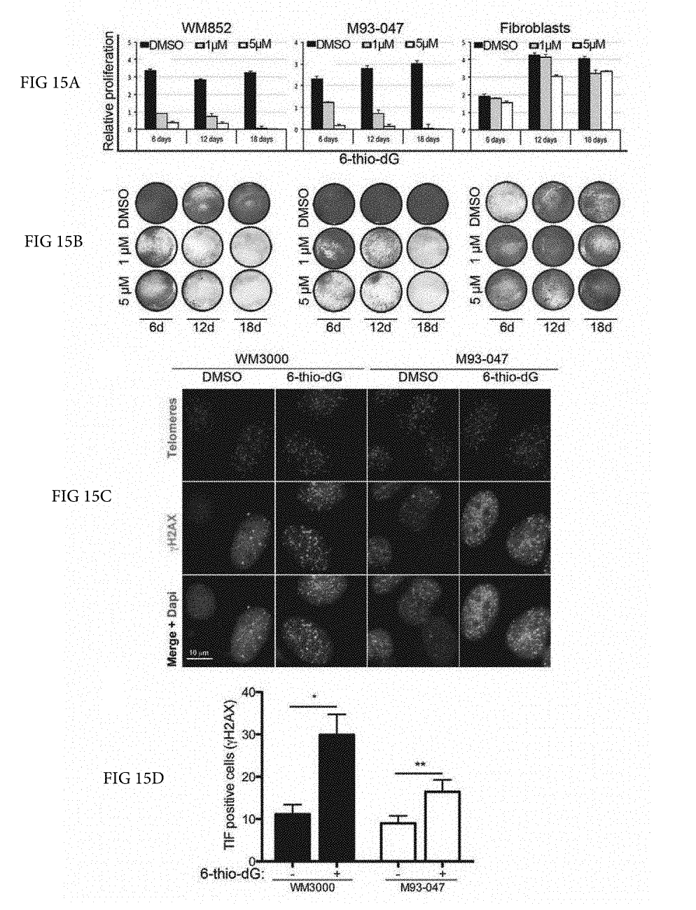

To determine the therapeutic value of exploiting melanoma cell dependency on TERT, we used the nucleoside analog 6-thio-2′-deoxyguanosie (6-Thio-dG; Mender et al., 2015). 6-thio-dG is a telomerase substrate precursor that is rapidly incorporated into the telomeres of cells expressing telomerase, acting as an uncapping agent and leading to rapid induction of TIFs³⁹. 6-thio-dG has no significant effects on red blood cells, white blood cells, alanine aminotransferase (ALT), aspartate aminotransferase (AST), and creatinine levels in mice³⁶. Treatment with 6-thio-dG impaired the viability of NRAS mutant melanoma cells (FIGS. 2a-b and FIG. 15a-b), including cells driven by secondary mutations in NRAS derived from melanoma patients with acquired resistance to BRAF and MEK inhibitors (FIG. 2b). As expected, 6-Thio-dG did not significantly affect telomerase negative cells such as non-transformed primary human melanocytes or fibroblasts (FIG. 2C), indicating selective activity in TERT-positive cells. Treatment of NRAS mutant melanoma cells with 6-Thio-dG led to cell death after 7-14 days (FIG. 2D), without evidence for significant telomere shortening after 14 days (FIG. 2E). Similar to the effects of TERT depletion, treatment with 6-Thio-dG led to increased levels of ROS (FIG. 2F) TIFs (FIG. 15C-D) and γH2AX (FIG. 2G), thus phenocopying the effects of TERT silencing. We next tested the effect of 6-Thio-dG-mediated telomere uncapping in NRAS mutant melanoma in an in vivo setting (FIG. 2H). Treatment of NRAS mutant bearing mice with 6-Thio-dG (2.5 mg/kg) slowed the growth of tumors, resulting in approximately 50% reduction in tumor volume after 14 days of treatment. The combined data indicate that 6-thio-dG triggers telomere damage (TIFs), increased ROS levels, and death of NRAS mutant melanoma cells in a time frame (7-14 days) that is markedly quicker that what has been reported for other tumors treated with telomerase inhibitors (Damm et al., 2001; Burchett et al., 2014).

C. Telomere Dysfunction is Coupled to Induction of an Anti-Oxidant Adaptive Response Program

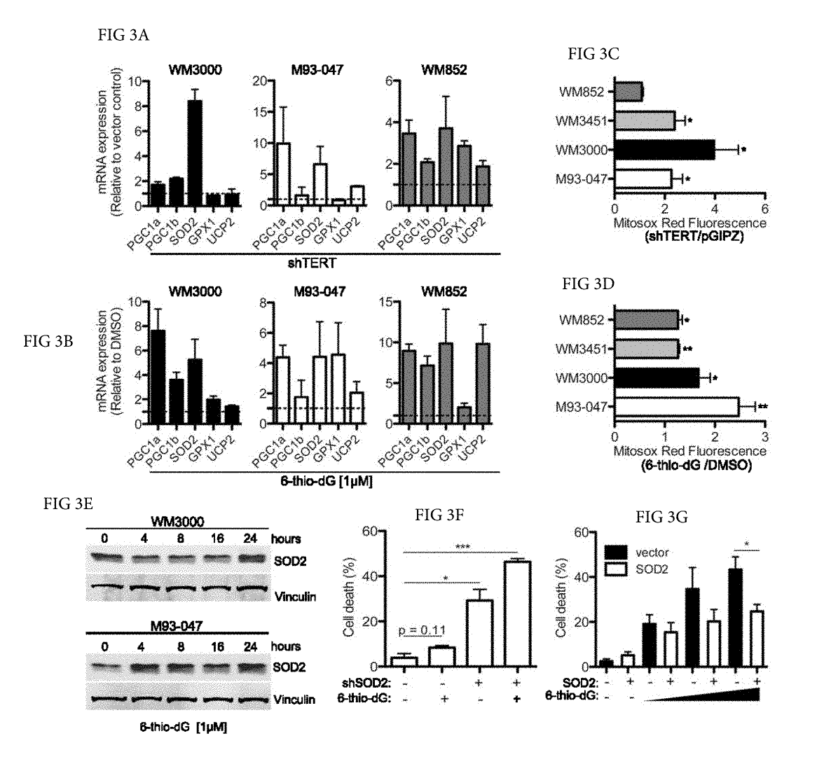

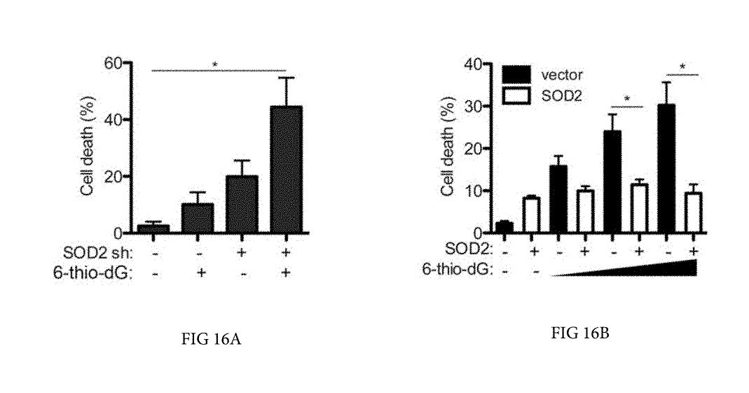

Increased ROS levels, such as those triggered by TERT depletion or treatment with 6-Thio-dG, can prompt the activation of an antioxidant enzyme-mediated adaptive response (Landriscina et al., 2009; Schumacker, 2015). Therefore, we evaluated whether the telomere dysfunction triggered by TERT depletion or treatment with 6-Thio-dG was associated with changes in the expression of antioxidant enzymes. TERT depletion or treatment with 6-thio-dG led to upregulation of several detoxifying enzymes such as SOD2, GPX1 and UCP2 (FIGS. 3A-B), as well as other enzymes involved in mitochondrial function and redox balance. Consistently, TERT depletion or treatment with 6-Thio-dG led to increased mitochondrial ROS levels (FIGS. 3C-D). We next sought to determine whether the most consistently upregulated anti-oxidant enzyme, SOD2 (FIGS. 3A, B and E), could be counteracting oxidative stress associated with telomere uncapping, enabling tumor cells to survive with high levels of ROS. Depletion of SOD2 enhanced the ability of 6-Thio-dG to induce apoptosis of NRAS mutant melanoma cells (FIGS. 3F and FIG. 16A). Conversely, overexpression of SOD2 attenuated the apoptosis-inducing effects of 6-Thio-dG (FIGS. 3G and FIG. 16B). These data suggest that TERT depletion or 6-Thio-dG-mediated telomere dysfunction leads to increased ROS levels, and that the subsequent activation of an anti-oxidant response could be targeted to improve the efficacy of telomerase-based anti-melanoma strategies.

D. Co-Targeting the Mitochondria Potentiates the Anti-Melanoma Effects of 6-Thio-DG.

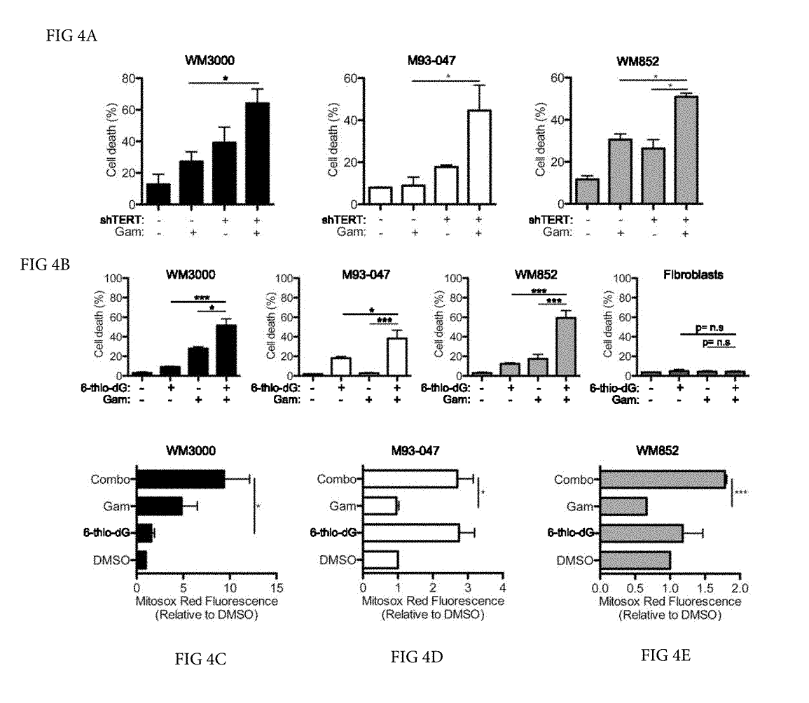

Based on the results described above, we hypothesized that combining a mitochondria disrupting agent with 6-Thio-dG or TERT depletion could effectively kill NRAS mutant melanoma cells by concurrently inducing high levels of ROS and blocking the anti-oxidant adaptive program induced by telomere dysfunction. We tested this hypothesis using Gamitrinib, an ATPase antagonist that disrupts mitochondrial function by selectively inhibiting mitochondrial Hsp90 (Kang et al., 2010). Gamitrinib has been shown to be a selective “mitochondriotoxic” agent in cancer cells, triggering rapid and complete loss of mitochondrial membrane potential and irreversible mitochondrial dysfunction in prostate, breast and lung cancer, as well as in BRAF-V600E-mutant melanoma (Chae et al., 2012). Importantly, Gamitrinib has been shown to downregulate SOD2 levels (Siegelin et al., 2011). We found that depletion of TERT enhanced the cytotoxic effects of Gamitrinib (FIG. 4A). We next treated NRAS mutant melanoma cells with 6-Thio-dG for 5 days to induce telomere dysfunction and increase ROS levels, as well as trigger an adaptive anti-oxidant response. NRAS mutant melanoma cells were then treated for 2 additional days with a combination of 6-Thio-dG and Gamitrinib. Pre-treatment of NRAS mutant melanoma cells with 6-Thio-dG sensitized cells to the cytotoxic effects of Gamitrinib, enhancing cell death as indicated by increased Annexin V and propidium iodide staining (FIG. 4B). Notably, this combination had minimal effects on non-transformed cells, such as human primary fibroblasts (FIG. 4B). Additionally, 6-Thio-dG in combination with Gamitrinib led to increased levels of mitochondrial ROS in NRAS mutant melanoma cells (FIGS. 4C-E).

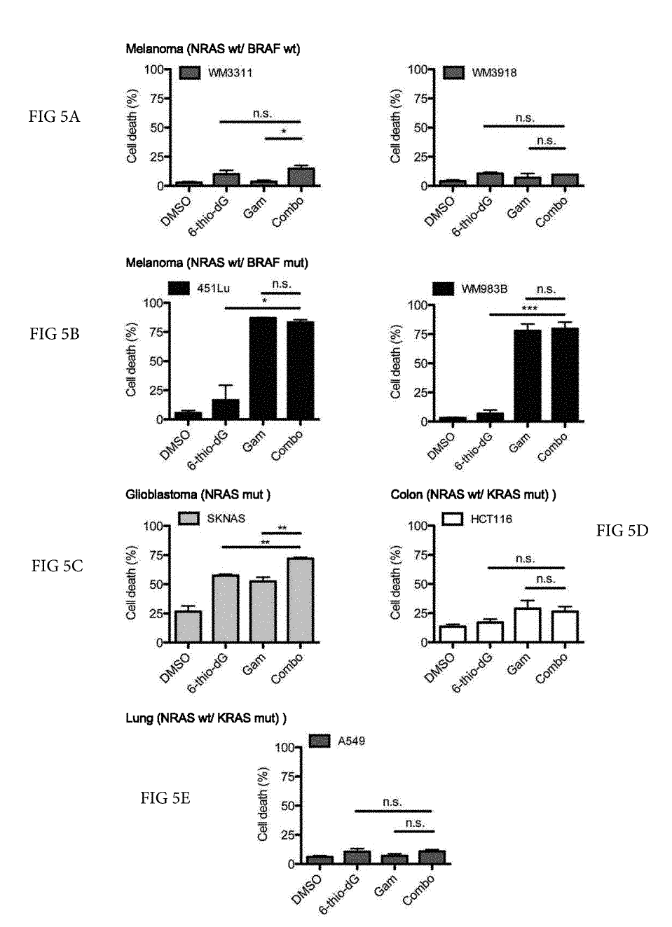

To determine if Gamitrinib could potentiate the efficacy of 6-thio-dG in melanoma cells lacking NRAS mutations, we treated NRAS wild-type melanoma cells with the nucleoside analog and the mitochondrial inhibitor as single agents or in combination (FIG. 5). We found that melanoma cells lacking NRAS or BRAF mutations (WT/WT) were resistant to 6-thio-dG and Gamitrinib as single agents or in combination, as these compounds did not induce cell death (FIG. 5a). As we have previously shown, BRAF-mutant melanoma cells were highly sensitive to Gamitrinib 6; while Gamitrinib alone led to 75-80% cell death, the combination with 6-thio-dG did not further enhanced death of BRAF mutant melanoma cells (FIG. 5b). As the aforementioned data indicated that Gamitrinib selectively potentiates the efficacy of 6-thio-dG in NRAS mutant melanoma cells, we wondered if this combination could trigger cell death in other RAS mutant tumor cells. To answer this question, we tested the effects of Gamitrinib and 6-thio-dG in KRAS mutant lung and colon cancer cell lines as well as NRAS mutant neuroblastoma cells. Interestingly, 6-thio-dG and Gamitrinib triggered significant death of NRAS mutant SKNAS neuroblastoma cells, as single agents or in combination (FIG. 5c). In contrast, 6-thio-dG and Gamitrinib did not induce substantial death of KRAS mutant HCT116 and A549 cells (FIG. 5d-e). These data suggest that Gamitrinib potentiates the efficacy of 6-thio-dG selectively in NRAS mutant tumor cells.

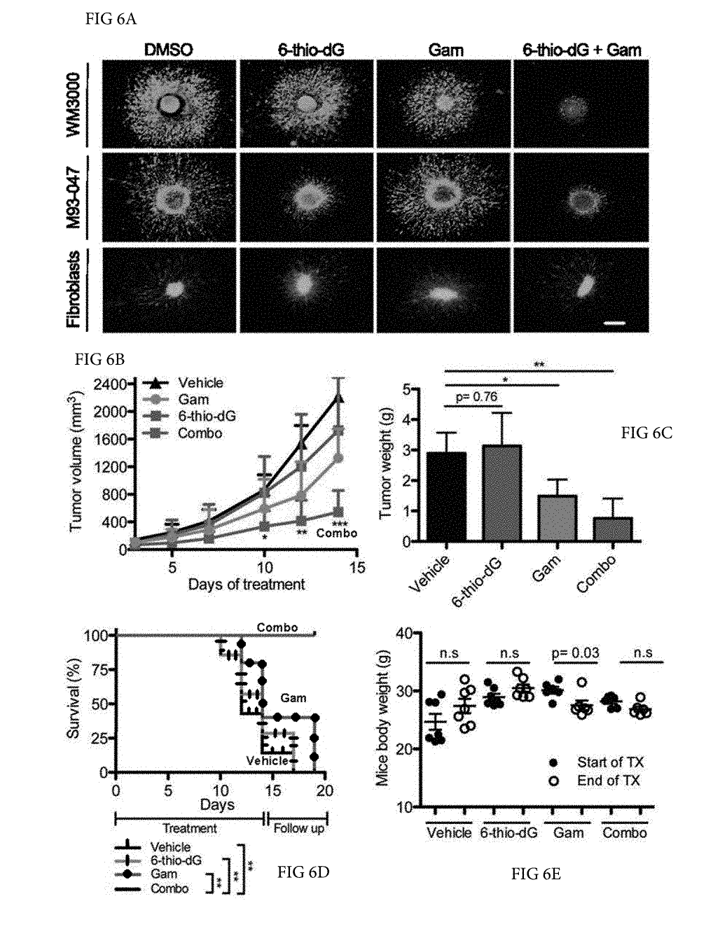

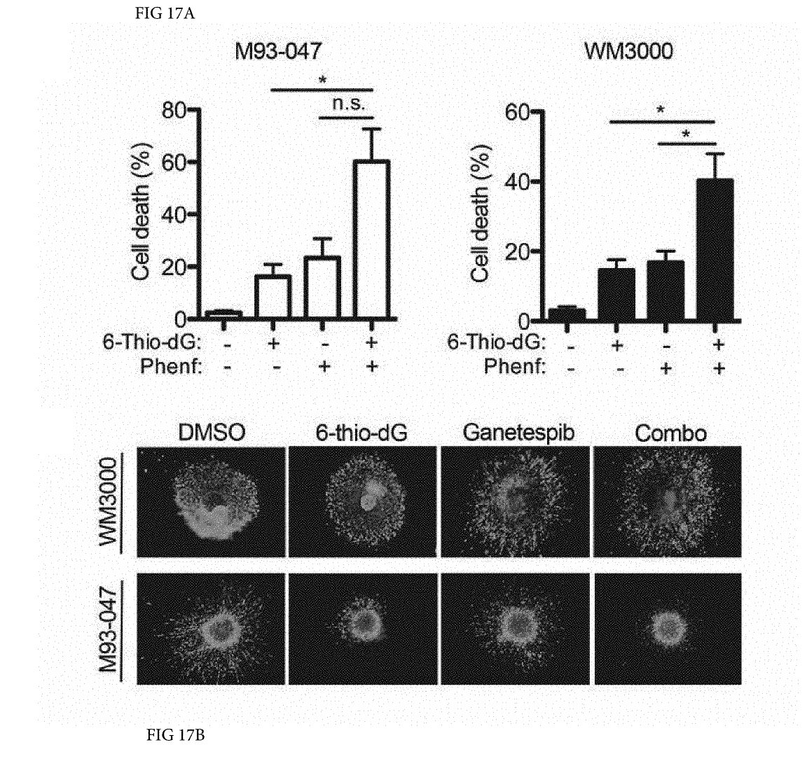

To further evaluate the efficacy of 6-Thio-dG in combination with Gamitrinib, we treated NRAS mutant melanoma cells grown as 3D-collagen embedded spheroids, which more closely mimic the in vivo behavior of melanoma. Consistent with our previous results, pretreatment of NRAS mutant melanoma spheroids with 6-thio-dG markedly potentiated the effect of Gamitrinib, and induced apoptosis of 3D melanoma spheroids (FIG. 6A). Of note, whereas treatment with another mitochondrial inhibitor phenformin resulted in similar cooperativity with 6-Thio-dG (FIG. 17A), treatment with ganetespib, an inhibitor of cytosolic HSP90, did not enhance the efficacy of 6-Thio-dG (FIG. 17B). These data support the premise that drug-induced mitochondrial impairment potentiates the effects of 6-Thio-dG.

We further explored the efficacy of this combination in vivo. To do this, NRAS mutant tumor bearing mice were treated with 6-Thio-dG (2.5 mg/kg ip qod) or Gamitrinib (12.5mg/Kg ip qd) as single agents, or in combination. Mice treated with the combination of 6-Thio-dG plus Gamitrinib exhibited significantly smaller tumors, compared to mice treated with either single agent (FIG. 6B-C). The combination of 6-Thio-dG plus Gamitrinib substantially prolonged the survival of tumor bearing mice (FIG. 6D), even after treatment withdrawal. Of note, this combination was well tolerated in vivo and did not significantly affect animal weight (FIG. 6e). Together our results indicate that therapeutic approaches exploiting melanoma cell addiction to TERT and combating the ability of melanoma to offset oxidative stress would render cancer cells vulnerable to drug-induced cell death.

All patents, patent applications and other references cited in this specification are hereby incorporated by reference in their entirety, including those listed in the references section below.

### REFERENCES

1. Artandi, S. E., and DePinho, R. A. (2010). Telomeres and telomerase in cancer. Carcinogenesis 31, 9-18.
2. Baerlocher, G. M., Oppliger Leibundgut, E., Ottmann, O. G., Spitzer, G., Odenike, O., McDevitt, M. A., Roth, A., Daskalakis, M., Burington, B., Stuart, M., et al. (2015). Telomerase Inhibitor Imetelstat in Patients with Essential Thrombocythemia. The New England journal of medicine 373, 920-928.
3. Bagheri, S., Nosrati, M., Li, S., Fong, S., Torabian, S., Rangel, J., Moore, D. H., Federman, S., Laposa, R. R., Baehner, F. L., et al. (2006). Genes and pathways downstream of telomerase in melanoma metastasis. Proceedings of the National Academy of Sciences of the United States of America 103, 11306-11311.
4. Bryan, C., Rice, C., Hoffman, H., Harkisheimer, M., Sweeney, M., and Skordalakes, E. (2015). Structural Basis of Telomerase Inhibition by the Highly Specific BIBR1532. Structure 23, 1934-1942.
5. Chae, Y. C., Caino, M. C., Lisanti, S., Ghosh, J. C., Dohi, T., Danial, N. N., Villanueva, J., Ferrero, S., Vaira, V., Santambrogio, L., et al. (2012). Control of tumor bioenergetics and survival stress signaling by mitochondrial HSP90s. Cancer cell 22, 331-344.
6. Choi, J., Southworth, L. K., Sarin, K. Y ., Venteicher, A. S., Ma, W., Chang, W., Cheung, P., Jun, S., Artandi, M. K., Shah, N., et al. (2008). TERT promotes epithelial proliferation through transcriptional control of a Myc- and Wnt-related developmental program. PLoS Genet 4, e10).
7. Damm, K., Hemmann, U., Garin-Chesa, P., Hauel, N., Kauffmann, I., Priepke, H., Niestroj, C., Daiber, C., Enenkel, B., Guilliard, B., et al. (2001). A highly selective telomerase inhibitor limiting human cancer cell proliferation. The EMBO journal 20, 6958-6968.
8. Easty, D. J., Gray, S. G., O'Byrne, K. J., O'Donnell, D., and Bennett, D. C. (2011). Receptor tyrosine kinases and their activation in melanoma. Pigment cell & melanoma research.
9. Garcia-Beccaria, M., Martinez, P., Mendez-Pertuz, M., Martinez, S., Blanco-Aparicio, C., Canamero, M., Mulero, F., Ambrogio, C., Flores, J. M., Megias, D., et al. (2015). Therapeutic inhibition of TRF1 impairs the growth of p53-deficient K-RasG12V-induced lung cancer by induction of telomeric DNA damage. EMBO molecular medicine 7, 930-949.
10. Griewank, K. G., Murali, R., Puig-Butille, J. A., Schilling, B., Livingstone, E., Potrony, M., Carrera, C., Schimming, T., Moller, I., Schwamborn, M., et al. (2014). TERT promoter mutation status as an independent prognostic factor in cutaneous melanoma. Journal of the National Cancer Institute 106.
11. Gupta, R., Dong, Y., Solomon, P. D., Wettersten, H. I., Cheng, C. J., Min, J. N., Henson, J., Dogra, S. K., Hwang, S. H., Hammock, B. D., et al. (2014). Synergistic tumor suppression by combined inhibition of telomerase and CDKN1A. Proceedings of the National Academy of Sciences of the United States of America 111, E3062-3071.
12. Haq, R., Shoag, J., Andreu-Perez, P., Yokoyama, S., Edelman, H., Rowe, G. C., Frederick, D. T., Hurley, A. D., Nellore, A., Kung, A. L., et al. (2013). Oncogenic BRAF regulates oxidative metabolism via PGC1alpha and MITF. Cancer cell 23, 302-315.
13. Hayward, N. K., Wilmott, J. S., Waddell, N., Johansson, P. A., Field, M. A., Nones, K., Patch, A. M., Kakavand, H., Alexandrov, L. B., Burke, H., et al. (2017). Whole-genome landscapes of major melanoma subtypes. Nature.
14. Hodis, E., Watson, I. R., Kryukov, G. V., Arold, S. T., Imielinski, M., Theurillat, J. P., Nickerson, E., Auclair, D., Li, L., Place, C., et al. (2012). A landscape of driver mutations in melanoma. Cell 150, 251-263.
15. Horn, S., Figl, A., Rachakonda, P. S., Fischer, C., Sucker, A., Gast, A., Kadel, S., Moll, I., Nagore, E., Hemminki, K., et al. (2013). TERT promoter mutations in familial and sporadic melanoma. Science 339, 959-961.
16. Hrdlickova, R., Nehyba, J., and Bose, H. R., Jr. (2012). Alternatively spliced telomerase reverse transcriptase variants lacking telomerase activity stimulate cell proliferation. Molecular and cellular biology 32, 4283-4296.
17. Hu, J., Hwang, S. S., Liesa, M., Gan, B., Sahin, E., Jaskelioff, M., Ding, Z., Ying, H., Boutin, A. T., Zhang, H., et al. (2012). Antitelomerase therapy provokes ALT and mitochondrial adaptive mechanisms in cancer. Cell 148, 651-663.
18. Huang, F. W., Hodis, E., Xu, M. J., Kryukov, G. V., Chin, L., and Garraway, L. A. (2013). Highly recurrent TERT promoter mutations in human melanoma. Science 339, 957-959.
19. Indran, I. R., Hande, M. P., and Pervaiz, S. (2011). hTERT overexpression alleviates intracellular ROS production, improves mitochondrial function, and inhibits ROS-mediated apoptosis in cancer cells. Cancer research 71, 266-276.
20. Jafri, M. A., Ansari, S. A., Alqahtani, M. H., and Shay, J. W. (2016). Roles of telomeres and telomerase in cancer, and advances in telomerase-targeted therapies. Genome Med 8, 69.
21. Jakob, J. A., Bassett, R. L., Jr., Ng, C. S., Curry, J. L., Joseph, R. W., Alvarado, G. C., Rohlfs, M. L., Richard, J., Gershenwald, J. E., Kim, K. B., et al. (2012). NRAS mutation status is an independent prognostic factor in metastatic melanoma. Cancer 118, 4014-4023.
22. Johnson, D. B., and Puzanov, I. (2015). Treatment of NRAS-mutant melanoma. Current treatment options in oncology 16, 15.
23. Johnson, D. B., Smalley, K. S., and Sosman, J. A. (2014). Molecular pathways: targeting NRAS in melanoma and acute myelogenous leukemia. Clinical cancer research: an official journal of the American Association for Cancer Research 20, 4186-4192.
24. Joseph, I., Tressler, R., Bassett, E., Harley, C., Buseman, C. M., Pattamatta, P., Wright, W. E., Shay, J. W., and Go, N. F. (2010). The telomerase inhibitor imetelstat depletes cancer stem cells in breast and pancreatic cancer cell lines. Cancer research 70, 9494-9504.
25. Kang, B. H., Siegelin, M. D., Plescia, J., Raskett, C. M., Garlick, D. S., Dohi, T., Lian, J. B., Stein, G. S., Languino, L. R., and Altieri, D. C. (2010). Preclinical characterization of mitochondria-targeted small molecule hsp90 inhibitors, gamitrinibs, in advanced prostate cancer. Clinical cancer research : an official journal of the American Association for Cancer Research 16, 4779-4788.
26. Koh, C. M., Khattar, E., Leow, S. C., Liu, C. Y., Muller, J., Ang, W. X., Li, Y., Franzoso, G., Li, S., Guccione, E., et al. (2015). Telomerase regulates MYC-driven oncogenesis independent of its reverse transcriptase activity. The Journal of clinical investigation 125, 2109-2122.
27. Kops, G. J., Dansen, T. B., Polderman, P. E., Saarloos, I., Wirtz, K. W., Coffer, P. J., Huang, T. T., Bos, J. L., Medema, R. H., and Burgering, B. M. (2002). Forkhead transcription factor FOXO3a protects quiescent cells from oxidative stress. Nature 419, 316-321.
28. Landriscina, M., Maddalena, F., Laudiero, G., and Esposito, F. (2009). Adaptation to oxidative stress, chemoresistance, and cell survival. Antioxid Redox Signal 11, 2701-2716.
29. Li, Y., Cheng, H. S., Chng, W. J., and Tergaonkar, V. (2016). Activation of mutant TERT promoter by RAS-ERK signaling is a key step in malignant progression of BRAF-mutant human melanomas. Proceedings of the National Academy of Sciences of the United States of America 113, 14402-14407.
30. Li, Y., and Tergaonkar, V. (2014). Noncanonical functions of telomerase:

implications in telomerase-targeted cancer therapies. Cancer research 74, 1639-1644.
31. Listerman, I., Sun, J., Gazzaniga, F. S., Lukas, J. L., and Blackburn, E. H. (2013). The major reverse transcriptase-incompetent splice variant of the human telomerase protein inhibits telomerase activity but protects from apoptosis. Cancer research 73, 2817-2828.
32. Maertens, O., Johnson, B., Hollstein, P., Frederick, D. T., Cooper, Z. A., Messiaen, L., Bronson, R. T., McMahon, M., Granter, S., Flaherty, K., et al. (2013). Elucidating distinct roles for NF1 in melanomagenesis. Cancer discovery 3, 338-349.
33. Masutomi, K., Possemato, R., Wong, J. M., Currier, J. L., Tothova, Z., Manola, J. B., Ganesan, S., Lansdorp, P. M., Collins, K., and Hahn, W. C. (2005). The telomerase reverse transcriptase regulates chromatin state and DNA damage responses. Proceedings of the National Academy of Sciences of the United States of America 102, 8222-8227.
34. Mender, I., Gryaznov, S., Dikmen, Z. G., Wright, W. E., and Shay, J. W. (2015). Induction of telomere dysfunction mediated by the telomerase substrate precursor 6-thio-2′-deoxyguanosine. Cancer discovery 5, 82-95.
35. Mender, I., and Shay, J. W. (2015). Telomerase Repeated Amplification Protocol (TRAP). Bio Protoc 5.
36. Nazarian, R., Shi, H., Wang, Q., Kong, X., Koya, R. C., Lee, H., Chen, Z., Lee, M. K., Attar, N., Sazegar, H., et al. (2010). Melanomas acquire resistance to B-RAF(V600E) inhibition by RTK or N-RAS upregulation. Nature 468, 973-977.
37. Nissan, M. H., Pratilas, C. A., Jones, A. M., Ramirez, R., Won, H., Liu, C., Tiwari, S., Kong, L., Hanrahan, A. J., Yao, Z., et al. (2014). Loss of NF1 in cutaneous melanoma is associated with RAS activation and MEK dependence. Cancer research 74, 2340-2350.
38. Nosrati, M., Li, S., Bagheri, S., Ginzinger, D., Blackburn, E. H., Debs, R. J., and Kashani-Sabet, M. (2004). Antitumor activity of systemically delivered ribozymes targeting murine telomerase RNA. Clinical cancer research : an official journal of the American Association for Cancer Research 10, 4983-4990.
39. Olmos, Y., Valle, I., Borniquel, S., Tierrez, A., Soria, E., Lamas, S., and Monsalve, M. (2009). Mutual dependence of Foxo3a and PGC-1alpha in the induction of oxidative stress genes. The Journal of biological chemistry 284, 14476-14484.
40. Postow, M. A., and Chapman, P. B. (2017). A step forward for patients with NRAS-mutant melanoma. The Lancet Oncology 18, 414-415.
41. Puri, N., Eller, M. S., Byers, H. R., Dykstra, S., Kubera, J., and Gilchrest, B. A. (2004). Telomere-based DNA damage responses: a new approach to melanoma. FASEB journal: official publication of the Federation of American Societies for Experimental Biology 18, 1373-1381.
42. Queisser, A., Heeg, S., Thaler, M., von Werder, A., and Opitz, O. G. (2013). Inhibition of telomerase induces alternative lengthening of telomeres during human esophageal carcinogenesis. Cancer Genet 206, 374-386.
43. Roesch, A., Vultur, A., Bogeski, I., Wang, H., Zimmermann, K. M., Speicher, D., Korbel, C., Laschke, M. W., Gimotty, P. A., Philipp, S. E., et al. (2013). Overcoming Intrinsic Multidrug Resistance in Melanoma by Blocking the Mitochondrial Respiratory Chain of Slow-Cycling JARID1 B(high) Cells. Cancer cell 23, 811-825.
44. Sarin, K.Y., Cheung, P., Gilison, D., Lee, E., Tennen, R. I., Wang, E., Artandi, M. K., Oro, A. E., and Artandi, S. E. (2005). Conditional telomerase induction causes proliferation of hair follicle stem cells. Nature 436, 1048-1052.
45. Schumacker, P. T. (2015). Reactive oxygen species in cancer: a dance with the devil. Cancer cell 27, 156-157.
46. Sharma, N. K., Reyes, A., Green, P., Caron, M. J., Bonini, M. G., Gordon, D. M., Holt, I. J., and Santos, J. H. (2012). Human telomerase acts as a hTR-independent reverse transcriptase in mitochondria. Nucleic acids research 40, 712-725.
47. Shay, J. W. (2016). Role of Telomeres and Telomerase in Aging and Cancer. Cancer discovery.
48. Shin, K. H., Kang, M. K., Dicterow, E., Kameta, A., Baluda, M. A., and Park, N. H. (2004). Introduction of human telomerase reverse transcriptase to normal human fibroblasts enhances DNA repair capacity. Clinical cancer research: an official journal of the American Association for Cancer Research 10, 2551-2560.
49. Siegelin, M. D., Dohi, T., Raskett, C. M., Orlowski, G. M., Powers, C. M., Gilbert, C. A., Ross, A. H., Plescia, J., and Altieri, D. C. (2011). Exploiting the mitochondrial unfolded protein response for cancer therapy in mice and human cells. The Journal of clinical investigation 121, 1349-1360.
50. Smith, L. L., Coller, H. A., and Roberts, J. M. (2003). Telomerase modulates expression of growth-controlling genes and enhances cell proliferation. Nature cell biology 5, 474-479.
51. Tefferi, A., Lasho, T. L., Begna, K. H., Patnaik, M. M., Zblewski, D. L., Finke, C. M., Laborde, R. R., Wassie, E., Schimek, L., Hanson, C. A., et al. (2015). A Pilot Study of the Telomerase Inhibitor Imetelstat for Myelofibrosis. The New England journal of medicine 373, 908-919.
52. Vallarelli, A. F., Rachakonda, P. S., Andre, J., Heidenreich, B., Riffaud, L., Bensussan, A., Kumar, R., and Dumaz, N. (2016). TERT promoter mutations in melanoma render TERT expression dependent on MAPK pathway activation. Oncotarget 7, 53127-53136.
53. Van Allen, E. M., Wagle, N., Sucker, A., Treacy, D. J., Johannessen, C. M., Goetz, E. M., Place, C. S., Taylor-Weiner, A., Whittaker, S., Kryukov, G. V., et al. (2014). The genetic landscape of clinical resistance to RAF inhibition in metastatic melanoma. Cancer discovery 4, 94-109.
54. Wong, D. J., and Ribas, A. (2016). Targeted Therapy for Melanoma. Cancer Treat Res 167, 251-262.
55. Zhang, G., Frederick, D. T., Wu, L., Wei, Z., Krepler, C., Srinivasan, S., Chae, Y. C., Xu, X., Choi, H., Dimwamwa, E., et al. (2016). Targeting mitochondrial biogenesis to overcome drug resistance to MAPK inhibitors. The Journal of clinical investigation 126, 1834-1856.

1. A method of treating cancer comprising (i) inhibiting telomerase and (ii) decreasing or downregulating anti-oxidant response or increasing oxidative stress.

2. A method of treating cancer comprising increasing telomere dysfunction and decreasing or downregulating anti-oxidant response or increasing oxidative stress.

3. The method of claim 1, wherein telomerase is inhibited or telomere dysfunction is increased via administration of 6-Thio-dG.

4. The method of claim 1, wherein anti-oxidant response is decreased or downregulated via administration of a mitochondria disrupting agent.

5. The method of claim 4, wherein the mitochondria disrupting agent is Gamitrinib.

6. The method of claim 1, wherein anti-oxidant response is decreased or downregulated via administration of an Hsp90 inhibitor which targets mitochondrial Hsp90.

7. The method of claim 1, wherein the cancer is an NRAS mutant cancer.

8. The method of claim 7, wherein the NRAS mutant cancer is melanoma or neuroblastoma.

9. The method of claim 1, further comprising administering an additional therapy.

10. The method of claim 9, wherein the additional therapy is selected from a chemotherapeutic agent, surgical removal of a malignancy, and radiation therapy.

11. The method of claim 10, wherein the chemotherapeutic agent is selected from imiquimod, trametinib (Mekinist) and cobimetinib, imatinib (Gleevec) and nilotinib, nivolumab [Opdivo], ipilimumab [Yervoy], and interferon.

12. A composition comprising a telomerase inhibitor and an agent that decreases or downregulates anti-oxidant response or induces oxidative stress.

13. The composition according to claim 12, wherein the telomerase inhibitor is an agent that decreases or downregulates TERT or blocks the reverse transcriptase activity of telomerase.

14. The composition according to claim 13, wherein the telomerase inhibitor is 6-Thio-dG.

15. The composition according to claim 12, wherein the telomerase inhibitor is selected from zidovudine, stavudine, tenofovir, didanosine, abacavir, TMPI, telomestatin, RHPS4, BRACO-19, TMPyP4, tertomotide, imetelstat sodium, ASTVAC-1, GX-301, UCPVax, UV-1, Vx-001, Vx-006, INO-1400, INVAC-1, ASTVAC-2, Telin(ab 4,4-dichloro-1-(2,4-dichlorophenyl)-3-methyl-5-pyrazolone), Vbx-011, Vbx-021, Vbx-026INO-5401, KML-001, TK-005, Ribovax, Vbx-016, ZI-HX, ZI-H04, and ZIH-03.

16. The composition according to claim 12, wherein the agent that inhibits an anti-oxidant response is a mitochondria disrupting agent.

17. The composition according to claim 16, wherein the mitochondria disrupting agent is Gamitrinib.

18. The composition according to claim 12, wherein the agent that inhibits an anti-oxidant response is an Hsp90 inhibitor which targets mitochondrial Hsp90.

19. The composition according to claim 16, wherein the mitochondrial disrupting agent is an HSP70 inhibitor.

20. The composition according to claim 16, wherein the mitochondrial disrupting agent is selected from phenformin, Pet-16, Lonidamine, Betulinic Acid, GSAO ((4-[N-[S-glutathionylacetyl]amino]phenylarsenoxide), Retinoid-related compound, Motexafin gadolinium (gadolinium texaphyrin), Menadione (2-methyl-1,4-naphthoquinone), diamide (diazenediacarboxylic acid bis 5N,N-dimethylamide), bismaleimido-hexane (BMH), dithiodipyridine (DTDP), Beta-lapachone, Butathione sulphoximine, Elesclomol sodium, imexon, ABT-737, ABT-263, Gossypol, A-385358, Obatoclax, 3-bromopyruvate, metformin and 2-deoxy-d-glucose.
